(19) United States
(12) Patent Application Publication
(10) Pub. No.: US 2019/0112356 A1
(43) Pub. Date: Apr. 18, 2019

(54) HIGH-AFFINITY IMMUNOPOLYMERS

(71) Applicants: CELL IDX, INC., San Diego, CA (US); THE UNIVERSITY OF CHICAGO

(72) Inventors: David A. SCHWARTZ, Encinitas, CA (US); Stephen J. KRON, Oak Park, IL (US); Amy C. FLOR, Chicago, IL (US)

(21) Appl. No.: 15/525,061

(22) PCT Filed: Nov. 6, 2015
(86) PCT No.: PCT/US2015/059424

§ 371 Date: May 6, 2017

Related U.S. Application Data
(60) Provisional application No. 62/076,430, filed on Nov. 6, 2014.

(51) Int. Cl. C07K 16/00; G01N 33/533; G01N 33/68

(52) U.S. Cl. CPC C07K 16/00; C07K 2317/92; G01N 33/6854; G01N 33/533

(57) ABSTRACT

The present disclosure is directed to high-affinity immunopolymers for use in a variety of immunoassays and other techniques. The immunopolymers comprise a plurality of antibodies, a plurality of coupling proteins, and a plurality of detectable labels. The plurality of antibodies and plurality of coupling proteins are associated by a high-efficiency conjugation moiety, and may be used in highly multiplexed assays. Methods of preparing the high-affinity immunopolymers are provided. Also disclosed are methods of detecting an antigen that include reacting an antigen with a high-affinity immunopolymer of the disclosure. Reagent mixtures and methods for quantitative immunochemical assays are also provided. The mixtures and methods may include the high-affinity immunopolymers disclosed herein.

### CROSS-REFERENCE TO RELATED APPLICATION

This application claims the benefit of U.S. Provisional Application No. 62/076,430, filed on Nov. 6, 2014, the disclosure of which is incorporated herein by reference in its entirety.

### BACKGROUND OF THE INVENTION

The use of immunoassays, in particular the use of immunohistochemical (IHC) staining, is of critical importance in the analysis of pathological conditions, such as in the analysis abnormal cells, including cancerous tumor cells. In IHC, an immunoglobulin or antibody that recognizes a specific antigen that may be present in a diseased tissue is applied to a thin section of that tissue obtained by biopsy. The binding of the antibody to its cognate antigen is then detected within the tissue section, typically by imaging the distribution of a chromogenic enzymatic product that is produced by an enzyme, such as a peroxidase, that is colocalized with the immunoglobulin. Examining the distribution of enzymatic product in comparison to the distribution of histological stain enables evaluation of the distribution of the antigen in the tissue section. In other immunoassays, the antibody binding to antigen may be detected by other means including optical, electrical, or chemical signals. Specific antigens may characterize particular cellular events, for example infection, injury, cell proliferation, inflammation, or drug response. Immunoassays are also widely used in basic research to understand the distribution and localization of antigens that serve as biomarkers, such as proteins differentially expressed in different parts of a cell or biological tissue, and to identify and quantify those antigens in biochemical assays. Immunoassays are thus of use, for example, in blots, sandwich assays, immunosorbent assays, immunocytochemical assays, and other related methods. All of these methods could benefit from improved immunochemical reagents.

Current methods of tissue analysis in clinical pathology are essentially restricted to single-antigen determinations performed on a single microscope slide. Importantly, there is a one-to-one correspondence between an antibody and its antigen, allowing ready determination of the antigen by binding or lack of binding of an antibody. Where each antibody is linked to a peroxidase or other enzyme, the presence of an antigen can be determined by the amount and distribution of enzyme product on the respective tissue section as a proxy. However, there is frequently more than one antigen that must be evaluated to complete a particular analysis. For example, in breast cancer, to optimally match a therapy to each patient, a minimum antigenic profile of a biopsy specimen would include evaluation of the presence and abundance of at least three antigens in the malignant cells of the tumor: Her2/neu (Her2), estrogen receptor (ER), and progesterone receptor (PR). Performing the analysis would thus require an assay for each of the three distinct immunoglobulins, each of which is typically a monospecific monoclonal antibody that is capable of detecting only one of the three antigens. To examine the degree of binding of the three different immunoglobulins to the malignant cells using traditional assays, three different IHC tests would thus need to be performed on three different tissue sections derived from the same block of tumor material. In addition, with traditional enzyme-based assays, as are typically now used in routine tissue analysis, each slide must be evaluated by a pathologist using a qualitative scoring system to determine the presence or absence and the level of expression of a given antigen. The results from the three tests must then be combined to determine the profile, which will offer prognostic information and aid in selection of therapy. Improving the efficiency, accuracy, and reliability of such assays is of major importance in the field.

Because of the need for high levels of reproducibility across laboratories, immunohistochemical analysis typically relies on standard methods, such as enzymatic detection, and a few well-studied antigens, as recognized by well-characterized antibodies (Moriya et al. (2006) Med. Mol. Morphol. 39:8-13; Payne et al. (2008) Histopathology 52:82-90; Yeh and Mies (2008) Arch. Pathol. Lab. Med. 132:349-57). Although direct detection of a peroxidase associated with a primary antibody is used in some cases, indirect detection by peroxidase-linked secondary antibodies or through primary antibodies tagged with high-affinity small-molecule/binding protein pairs, such as biotin and avidin, can be used to amplify the signal and thus to improve sensitivity of the assays. In each of these cases, intensity of signal is typically judged subjectively, however, thus limiting the diagnostic and prognostic value of the assays.

About 1.6 million breast biopsies a year are performed in the United States, typically in women who have developed a breast lump. A biopsy entails sampling the tissue of the lump by fine needle aspiration or core needle through the skin or an open procedure. The resulting tissue is then examined to detect the presence of malignant cells. The majority of such biopsies are considered benign based on examination of the tissue using histology techniques. In 2010, histological analysis determined that 260,000 biopsies displayed malignancy. Of these, some 200,000 women had invasive breast cancer, and others were described as ductal carcinoma in situ (DCIS), in which cancer cells have not invaded the surrounding tissue. Advances in early detection and definitive treatment of primary tumors have dramatically improved breast cancer survival statistics. Yet, many tumors escape early detection or, in spite of effective primary therapy, go on to develop distant metastases, the leading cause of breast cancer mortality. Much of the current effort in molecular analysis of breast cancer is directed at identifying new biomarkers and defining the mechanistic determinants of prognosis and prediction. It would be helpful if any such novel disease markers could be readily incorporated into the routine immunohistochemical staining of tissue biopsies.

As mentioned above, because of the limitations of detection using enzyme conjugates, each target antigen is often evaluated on a separate histological section, and internal controls are not readily implemented. As a result, quantitation, evaluation of colocalization, and subcellular resolution are problematic. A well-established alternative to enzyme conjugates for detection in immunohistochemistry is fluorescent labeling. The principal advantage of this approach lies in the potential for multiplexing. In short, either the antibody itself, or more typically, a secondary antibody or other indirect detection reagent, is labeled with a fluorescent group, protein, or other material of known spectroscopic properties. Upon illumination of the sample with light at the excitation wavelength of the fluorescent label, the presence of a fluorescent signal at a specific emission wavelength, and the localization of that signal at sites within a tissue section are observed. The fluorescence signal thus serves the same purpose as the chromogenic enzyme product in providing information regarding the amount and distribution of the antigen.

Covalent modification of immunoglobulins with chemically-reactive fluorescent reagents to form fluorescent antibodies, and the use of the fluorescent antibodies in the detection of antigens is now well-established, having been demonstrated by Coons' modification of specific immunoglobulins with fluorescein isothiocyanate in 1941. Coons et al. (1941) Proc Soc Exp Biol. 47:200-2. Simultaneous detection of two antigens by antibodies labeled with distinct fluorescent colors, fluorescein and rhodamine, followed soon thereafter. Modern chemistry has provided a broad range of chemically-reactive fluorophores, with excitation and emission spectra that range from the ultraviolet to infrared. In turn, modern coating methods have produced interference filters that can readily distinguish four or more different fluorophores over the visible light spectrum, with signal-to-noise ratios much greater than 10, by selecting specific excitation and emission bands.

Fluorescence-based immunoassays are of growing importance in the staining of pathological sections and in cytometry, at least in part because of the ability to distinguish multiple antigens through the use of multiple, differentially-labeled, fluorescent antibodies. In these approaches, the different antibodies are distinguishable, for example, by measuring fluorescence emitted at different wavelengths. Other spectroscopic properties of the different fluorophores may also potentially be used to distinguish the bound antibodies. While it has been recognized that fluorescence-based assays could potentially be used to detect >3 antigens on a single tissue, current methods with fluorescently-labeled primary antibodies do not provide sufficient sensitivity. In particular, only 3-5 fluorophores can be conjugated to a single antibody due to fluorescence quenching or reduced immunoreactivity upon incorporation of >5 fluorophores into the antibody. Furthermore, monoclonal antibodies bind to a single epitope on any target antigen and thus further inhibit any amplification of signal by binding to multiple sites.

Fluorescently-labeled polyclonal secondary antibodies can produce a stronger signal than fluorescently-labeled primary antibodies, because multiple secondary antibodies can bind to distinct epitopes presented on each primary antibody molecule, but this approach is typically limited to the detection of only one or two targets, as the majority of monoclonal antibodies have been produced in only two species, i.e. mouse and rabbit.

One approach to enable multiplexing using antibodies from one species has been the use of hapten-modified antibodies and fluorescently labeled anti-hapten antibodies. However, using conventional reagents, this method yields a signal equal to or less intense than that generated by a fluorescently-labeled secondary antibody.

Another factor limiting the application of fluorescently-labeled antibodies to immunochemical assays is cost. Antibodies, in particular primary antibodies, are expensive to produce, and any modification or conjugation method must be extremely efficient, must generate an easily purified product, must be reproducible, and must be scalable.

To further advance immunohistochemistry there thus remains a need for a technology that is able to produce a panel of reagents that can satisfy some or all of the following criteria: 1) ability to analyze multiple antigens simultaneously within a single tissue sample and within the context of tissue morphology with greater sensitivity and specificity than currently available, 2) ability to analyze the spatial distribution of multiple antigens in relationship to each other, 3) ability to quantify each antigen individually and to determine ratios of one antigen to another with greater sensitivity and specificity, 4) ability to identify objects of interest (cell types) based on their staining patterns, 5) ability to numerically quantify objects of interest and 6) ability to be incorporated in automated staining and image analyses paradigms that will allow complete automation of the analyses of multiple antigens on a single tissue. Further, there remains a need for user-friendly kits enabling rapid, standardized multiple-antigen detection and quantification for diagnostic or research purposes. The present disclosure is directed to addressing these needs, as well as other problems in immunoassays that are not currently being addressed.

For example, U.S. Pat. No. 5,650,334 describes fluorescent labeling compositions comprising a linear polysaccharide backbone molecule having a plurality of target-binding molecules, such as antibodies or nucleic acids, attached to the backbone at spaced-apart intervals. Such compositions are purportedly useful in the detection of target substances in biological samples. The compositions allow for a multiplicity of target-binding molecules and a multiplicity of fluorescent labels within a single molecular entity, but the compositions are difficult to prepare efficiently in high yields and likely to result in heterogeneous products.

U.S. Pat. Nos. 6,303,757 and 6,303,758 describe highly-sensitive dye-labeled and polymerized antibodies. The antibody polymers can purportedly detect target substances on surfaces at low concentrations. The polymers are generated by the reaction of a solution of monomeric immunoglobulins with a polyfunctional crosslinking reagent, however, thus resulting in a heterogeneous product in low yields and with poor binding efficiency.

U.S. Pat. No. 4,046,871 describes compositions and serological methods relating to bovine serum albumin (BSA) polymers. Such compositions and methods are purportedly useful in potentiating agglutination reactions of IgGs. The BSA monomers are joined to one another through peptide bonds to form the polymers using a peptide bond forming reagent. The polymers are not, however, detectably labeled.

U.S. Pat. No. 4,657,853 describes covalent conjugates of polymerized enzyme and antibody. The conjugates purportedly provide enhanced signal generation, thus resulting in short assay time and high sensitivity. Polymerization of the enzyme is effected using poorly soluble and unstable bifunctional reagents, however, and the polymerization reaction is thus highly dependent on enzyme concentration and other reaction conditions and is thus difficult to optimize and control.

U.S. Pat. No. 5,543,332 describes reagents and conjugates comprising moieties derived from divinyl sulfone. The reagents and conjugates are based on water-soluble, polymeric carrier molecules having moderate to high molecular weight and to which are covalently coupled reactive divinyl-sulfone-derived moieties or bridging divinyl-sulfone-derived moieties. Divinyl sulfone is a homofunctional crosslinker that is not particularly reactive, however. The coupling reactions are therefore limited to carrier molecules capable of reacting with divinyl sulfone. They are thus inefficient, non-specific, and require relatively high concentrations of reactants.

U.S. Pat. No. 6,627,460 describes methods for preparing water-soluble cross-linked conjugates. The conjugates comprise a water-soluble carrier component, for example various polymers including natural and synthetic polysaccharides, homopoly (amino acid)s, natural and synthetic polypeptides and proteins, and synthetic polymers. Crosslinking is effected using bifunctional reagents. As was true with U.S. Pat. No. 4,657,853, the crosslinking reaction is thus highly dependent on enzyme concentration and other reaction conditions. It is therefore difficult to optimize and control.

U.S. Patent Publication No. 2007/0037138 A1 describes reference standards for use in immunohistochemistry, immunocytochemistry, and molecular cytogenetics. The reference standards comprise a support medium and a quantity of at least one detectable entity supported by the support medium. The standards are purportedly useful the quantification of signals obtained from immunohistochemical assays but require additional handling during tissue preparation and are not applicable to non-embedded samples.

Despite these attempts, there continues to be a need for the development of improved immunoassay staining reagents, methods, and kits that are more sensitive, more specific, and more able to detect multiple antigens in a single assay.

### SUMMARY OF THE INVENTION

The present disclosure addresses these and other needs by providing in one aspect a high-affinity immunopolymer that finds utility in a variety of immunochemical assays. Specifically, the immunopolymer of the disclosure comprises:
a plurality of antibodies;
a plurality of coupling proteins; and
a plurality of detectable labels;
wherein the plurality of antibodies and the plurality of coupling proteins are associated by a high-efficiency conjugation moiety.

In some immunopolymer embodiments, the plurality of detectable labels are linked either to the plurality of antibodies or to the plurality of coupling proteins. In other embodiments, the plurality of detectable labels are linked to the plurality of antibodies, are linked to the plurality of coupling proteins, or are linked both to the plurality of antibodies and to the plurality of coupling proteins.

In some embodiments, the high-efficiency conjugation moiety is a covalent conjugation moiety. In specific embodiments, the covalent conjugation moiety is a Schiff base, such as a hydrazone or an oxime. In other embodiments, the covalent conjugation moiety is formed by a click reaction.

In some embodiments, the high-efficiency conjugation moiety is a non-covalent conjugation moiety, such as an oligonucleotide hybridization pair or a protein-ligand binding pair.

In certain embodiments, the high-efficiency conjugation moiety is at least 50% efficient. More specifically, the high-efficiency conjugation moiety is at least 50% efficient at a protein concentration no more than 0.5 mg/mL.

In other embodiments, the immunopolymer comprises at least three antibodies. More specifically, each antibody in the at least three antibodies is the same.

In still other embodiments, the immunopolymer comprises at least three coupling proteins, and specifically each protein in the at least three coupling proteins is the same.

More specifically, the immunopolymer comprises at least three antibodies and at least three coupling proteins, and even more specifically each antibody in the at least three antibodies is the same, and each protein in the at least three coupling proteins is the same.

In certain embodiments, the immunopolymer has an average molecular weight of at least 200K.

In other embodiments, the immunopolymer comprises an average of at least 4 detectable labels.

In still other embodiments, each coupling protein in the plurality of coupling proteins is an albumin or an immunoglobulin.

In certain embodiments, each antibody in the plurality of antibodies is a primary antibody, a secondary antibody, or an anti-hapten antibody.

In some embodiments, each detectable label in the plurality of detectable labels is a fluorophore, an enzyme, an upconverting nanoparticle, or a quantum dot.

In another aspect, the disclosure provides methods of detecting an antigen, comprising:
reacting a first antigen with a first high-affinity immunopolymer, wherein the first high-affinity immunopolymer is an immunopolymer of the disclosure; and
detecting the first high-affinity immunopolymer.

In some embodiments, the first antigen is within a tissue section.

In other embodiments, the first antigen is in or on a cell.

In specific embodiments, the method further comprises the step of sorting cells that have bound the first immunopolymer.

In another aspect, the disclosure provides methods of preparing a high-affinity immunopolymer, comprising:
modifying an antibody with a first conjugating reagent;
modifying a coupling protein a second conjugating reagent; and
reacting the modified antibody with the modified coupling protein to generate the high-affinity immunopolymer;
wherein the first conjugating reagent and the second conjugating reagent associate with one another at high efficiency.

In yet another aspect, the disclosure provides reagent mixtures for calibration of an immunochemical assay, comprising:
a first population and a second population of particles comprising a detectable agent,
wherein the detectable agent is associated with the surface of the first population of particles at a first surface density and is associated with the surface of the second population of particles at a second surface density, and wherein the detectable agent is a cellular antigen or a hapten.

In still yet another aspect are provided methods for calibration of an immunochemical assay comprising:
treating a population of cells with a first population and a second population of particles comprising a detectable agent, wherein the detectable agent is associated with the surface of the first population of particles at a first surface density and is associated with the surface of the second population of particles at a second surface density, and wherein the detectable agent is a cellular antigen or a hapten.

The disclosure additionally provides, in another aspect, kits comprising the high-affinity immunopolymer of the disclosure; and
instructions for use in an immunoassay.

### BRIEF DESCRIPTION OF THE DRAWINGS

FIG. 1. Schematic representation of an exemplary synthesis of a [(BSA-fluorophore)-IgG] immunopolymer using hydrazone chemistry. (A) Free amino groups on BSA are modified with a limiting amount of an amino-reactive aldehyde linking reagent (sulfo-NHS-4FB). The BSA is then further reacted with an amino-reactive fluorophore (FL). (B) IgG amino groups are modified with an amino-reactive hydrazine linking reagent (S-HyNic). (C) Modified BSA-FL and modified IgG are reacted to generate a fluorescent hydrazone-linked high-affinity immunopolymer, [(BSA-FL)-IgG], with high efficiency.

FIG. 2. Hydrazone-linked immunopolymer staining. Progesterone receptor (PR)-positive breast cancer tissue was stained with anti-PR primary antibody from rabbit (Rb) followed by a [(BSA-Dy488)-anti-Rb] immunopolymer prepared as outlined in FIG. 1.

FIG. 3. Schematic representation of an exemplary synthesis of an [(IgG-BSA)-fluorophore] immunopolymer using oxime chemistry with a thiol-reactive maleimide oxyamino (MOA) linking reagent. (A) BSA amino groups are modified with an amino-reactive aldehyde linking reagent (sulfo-NHS-4FB), as in FIG. 1. (B) Disulfide groups on an IgG are reduced to generate free thiol groups. The thiol groups are then reacted with the maleimide group on MOA linking reagent to form a modified antibody. (C) Modified BSA and modified IgG are reacted to generate an oxime-linked high-affinity immunopolymer with high efficiency. The product of the reaction is labeled with an amino-reactive fluorophore to generate a fluorescent oxime-linked high-affinity immunopolymer, [(IgG-BSA)-FL].

FIG. 4. Oxime-linked immunopolymer staining. Her2+ breast cancer tissue was stained with an anti-Her2 primary rabbit antibody followed by treatment with an [(anti-Rb-BSA)-Dy550] immunopolymer prepared as outlined in FIG. 3.

FIG. 5A. Schematic representation of an exemplary synthesis of an [(IgG-BSA)-FL] immunopolymer using oxime chemistry with an amino-reactive oxyamino (AOA) linking reagent. (A) BSA amino groups are modified with an amino-reactive aldehyde linking reagent (sulfo-NHS-4FB). (B) IgG amino groups are modified with the amino-reactive group on the AOA linking reagent. (C) Modified BSA and modified IgG are reacted to generate an oxime-linked high-affinity immunopolymer with high efficiency. The immunopolymer is subsequently labeled with an amino-reactive fluorophore to generate a fluorescent oxime-linked high-affinity immunopolymer, [(IgG-BSA)-FL].

FIG. 5B. SDS-PAGE of the product of the reaction of FIG. 5A. Lane 1: MW standards; Lane 2: unreacted BSA; Lane 3: unreacted IgG; Lane 4: product of the polymerization reaction.

FIG. 6. Effect of increasing fluorophore incorporation on immunopolymer staining. Comparison of (A) conventional staining of estrogen receptor (ER) on ER positive (ER+) breast cancer tissue using monoclonal anti-ER primary antibody from rabbit (Rb) followed by staining with a conventional, directly-labeled, monomeric polyclonal anti-Rb-Dy650 antibody from goat; or (B-F) followed by staining with an [(anti-Rb-BSA)-Dy650] high-affinity immunopolymer having increasing levels of average fluorophore incorporation: (B) 4.2 (C) 6.6 (D) 10.1 (E) 14.1 (F) 16.4 fluorophores per immunopolymer. The immunopolymers were created using the AOA linking reagent.

FIG. 7. IHC results comparing the sensitivity of a conventional fluorescent secondary antibody to the novel fluorescent immunopolymers of the instant disclosure. Triple-positive (Her2+ER+PR+) breast cancer tissue was stained with either anti-Her2 (A, D), anti-ER (B, E), or anti-PR (C, F), each from rabbit (Rb). The top row (A-C) shows conventional secondary antibody staining using monomeric anti-Rb in three fluorescent colors (Dy550, Dy650, or Dy488). The lower row (D-F) shows secondary staining using novel immunopolymers [(anti-Rb-BSA)-FL)] in the respective three fluorescent colors. The immunopolymers were created using the AOA linking reagent.

FIG. 8. Schematic representation of an exemplary synthesis of a [(Poly(BSA)-FL)-IgG] immunopolymer. (A) BSA is separately labeled with a limiting amount of an amino-reactive aldehyde linking reagent (sulfo-NHS-4FB) and a limiting amount of an amino-reactive hydrazine linking reagent (S-HyNic). The FB-modified BSA is reacted with the HyNic-modified BSA to generate a hydrazone-linked BSA polymer (polyBSA) with remaining unreacted amino groups. (C) The polyBSA is labeled with a limiting amount of an amino-reactive fluorophore and is again modified with the amino-reactive aldehyde linking reagent. (D) IgG is modified with an amino-reactive hydrazine linking reagent and is added to the activated fluorescent polyBSA sample to generate a fluorescent oxime-linked [(Poly(BSA)-FL)-IgG] high-affinity immunopolymer.

FIG. 9. Enhanced immunofluorescence using three-step immunofluorescent staining. (A) ER+ breast cancer tissue was stained in two steps with a rabbit anti-ER antibody followed by a [(goat anti-rabbit-BSA)-Dy650] immunopolymer. (B) Enhanced staining is accomplished in three steps by staining the tissue of panel A with a third staining using an [(anti-goat-BSA)-Dy650] immunopolymer.

FIG. 10. Direct antigen staining using a fluorescent Poly(BSA) immunopolymer. ER+ breast cancer tissue was either (A) stained conventionally with an anti-ER rabbit antibody followed by a conventional anti-Rb-Dy650 secondary antibody; or (B) directly stained using a [(PolyBSA-Dy650)-anti-ER] immunopolymer prepared as shown in FIG. 8.

FIG. 11. IHC results illustrating the sensitivity of hapten-mediated antigen detection. Using biotin (BIO) as the hapten, PR+ breast cancer tissue was stained using the following combinations: (A) conventional non-hapten approach, using an unlabeled rabbit anti-PR antibody with a conventional anti-Rb-Dy488 secondary antibody; (B) conventional biotin/anti-biotin hapten approach, using an anti-PR-biotin antibody with a commercial anti-biotin-Dy488 secondary antibody; (C-F) hapten approach using a novel immunopolymer, [(anti-biotin-BSA)-Dy488]. A range of immunopolymer concentrations were tested: (C) 40 μg/mL (D) 20 μg/mL (E) 10 μg/mL and (F) 5 μg/mL. The lower intensity signal observed at higher immunopolymer concentrations (C) is ascribed to a quenching of the fluorescent signal from the immunopolymer at the higher concentrations.

FIG. 12. Hapten-mediated triple antigen staining. Triple-positive (Her2+ER+PR+) breast cancer tissue was stained with a cocktail of three hapten-labeled primary antibodies: anti-Her2-DNP (xHer2-DNP); anti-ER-digoxigenin (xER-DIG); and anti-PR-biotin (xPR-BIO). The sample was then stained with a cocktail of three fluorescently-labeled (anti-hapten-BSA) immunopolymers: [(xDNP-BSA)-Dy550], [(xDIG-BSA)-Dy650], and [(xBIO-BSA)-Dy488]. Results are shown presenting (A) Her2 staining in the Dy550 fluorescent channel; (B) ER staining in the Dy650 channel; and (C) PR staining in the Dy488 channel. The anti-hapten immunopolymers used here were created using the AOA conjugating reagent as shown in FIG. 5A.

FIG. 13. Schematic representation of an exemplary synthesis of [(Poly(IgG)-FL] immunopolymers using the AOA linking reagent. (A) Amino groups of a non-specific IgG are modified with an amino-reactive aldehyde linking reagent (sulfo-NHS-4FB); (B) Amino groups of a target-specific IgG are modified with the AOA linking reagent; (C) The modified IgG's are combined to generate an oxime-linked immunopolymer at high efficiency. The immunopolymer is subsequently labeled with amine-reactive fluorophores to generate the fluorescent oxime-linked immunopolymer, [Poly(IgG)-FL].

FIG. 14. [(Poly(IgG)-FL] immunopolymer staining. Staining of Her2 on Her2+ breast cancer tissue with rabbit anti-Her2 followed by [(anti-Rb-Bov(IgG))-Dy550] immunopolymer.

FIG. 15. Enzymatic IHC staining using diluted immunopolymers. ER+ breast cancer tissue was first stained using rabbit anti-ER followed by either (A) a commercial anti-rabbit polymer at 1× (˜0.5 mg/mL), as provided by manufacturer, or a diluted novel immunopolymer: (B) 1/50× dilution, ˜20 μg/mL; (C) 1/100× dilution; ˜10 μg/mL. Enzyme-labeled immunopolymers were prepared using the workflow shown in FIG. 5A, except that HRP was used in place of BSA.

FIG. 16. Immunostaining using a primary-antibody immunopolymer. Ki67+ breast cancer tissue was stained with primary [(anti-Ki67-poly(HRP)] immunopolymer prepared using the AOA linking reagent as described above. 3,3′-diaminobenzidine (DAB) chromogenic HRP enzyme substrate was then used to visualize HRP on tissue (dark grey). Nuclei were counterstained with hematoxylin (light grey).

FIG. 17. Protein immunoblotting with enzymatic immunopolymers. A standard ‘Western’ blot for Beta-actin in mouse cell lysate was prepared, comparing signal detection using (A) conventional anti-Rb-HRP to (B) [(anti-Rb-Poly(HRP)] or (C) [(anti-Rb-Poly(SBP)] immunopolymers. A decreasing amount of total protein was loaded per lane (10, 5, 1 μg/lane) to test the limits of detection.

FIG. 18. Comparison of tyramide signal amplification (TSA) using novel immunopolymer vs. conventional signal detection methods. All tissues were PR+ and were first stained with anti-PR (xPR, Rb). (A) Conventional detection using anti-rabbit-Dy650 (xRb-Dy650); (B) Conventional signal amplification strategy using biotin/streptavidin, applying first anti-rabbit-biotin (xRb-BIO) followed by streptavidin-Dy650 (SA-Dy650); (C) Conventional TSA strategy using commercial anti-rabbit-HRP (xRb-HRP), followed by commercial biotinylated tyramide (Tyr-BIO) and SA-Dy650; (D) TSA using immunopolymer [anti-Rb-poly(HRP)] followed by Tyr-BIO and SA-Dy650.

FIG. 19. Flow cytometry staining comparing anti-hapten immunopolymers to conventional staining approaches. Various immunoreagents were used to identify T cells (CD3+) within a heterogeneous population of human white blood cells. Rows A-C illustrate non-specific binding of immunoreagents to cells, a particular concern for staining of white blood cells. Rows D-F illustrate specific labeling of T cells by anti-CD3 and various secondary reagents. Rows A, D: a first group of cell samples were labeled by haptenylated antibody anti-CD3-DNP, followed by novel anti-DNP immunopolymer in each of three fluorescent colors (Dy488, Dy550, Dy650). Rows B, E: a second group of cell samples were stained in a conventional manner using unlabeled antibody anti-CD3 from mouse, followed by commercial anti-mouse secondary antibody in each of three fluorescent colors. Rows C, F: a third group of cell samples were labeled using a conventional biotin/streptavidin approach first with antibody anti-CD3-biotin, followed by fluorescent streptavidin in each of three fluorescent colors.

FIG. 20. (A) Preparation of haptenylated microspheres and (B) subsequent labeling using anti-hapten fluorescent immunopolymers.

FIG. 21. (A) Evaluation of labeled haptenylated microspheres by flow cytometry; (B) fluorescent microscope visualization of labeled microspheres.

FIG. 22. Pre-assembly strategy for use of haptenylated calibrant microspheres with similarly labeled tissue wherein: (A) microspheres modified with different densities of hapten are labeled with [(anti-hapten-BSA)-FL] immunopolymer prior to their transfer to the tissue sample; (B) the tissue sample is stained with a haptenylated primary antibody followed by treatment with [(anti-hapten-BSA)-FL] immunopolymer; and (C) the pre-labeled microspheres are added to labeled tissue sample. The target antigen is symbolized as a pentagon, and the hapten is symbolized as a square. Note that the number of conjugates shown in this scheme to be associated with a given microsphere or haptenylated primary antibody are for illustrative purposes only and should not be considered indicative of actual labeling levels.

FIG. 23. In situ self-assembly strategy for use of haptenylated calibrant microspheres with similarly labeled tissue wherein (A) haptenylated microspheres and haptenylated primary antibodies are added to the tissue sample together, and (B) [(anti-hapten-BSA)-FL] immunopolymer is subsequently added to the sample. As in FIG. 22, the target antigen is symbolized as a pentagon, the hapten is symbolized as a square, and the number of conjugates shown in the scheme to be associated with a given microsphere or haptenylated primary antibody are for illustrative purposes only and should not be considered indicative of actual labeling levels.

FIG. 24. Microscopic evaluation of haptenlyated microspheres used with similarly labeled tissue. (A) In fluorescent channel #1, the fluorescent tracking dye of the microspheres is visible, as well as nuclear counterstain DAPI. This information is used to enumerate all microspheres and all nuclei. (B) In fluorescent channel #2, ‘LO’ and ‘HI’ labeled calibrant microspheres are visible, as well as labeled cells of interest (here, PR+). This information is used to evaluate cellular signaling level, and enumerate cells of interest.

FIG. 25. Automated scoring of labeled tissue using calibrant micro spheres and open-source imaging software. (A) Software used to enumerate all nuclei in fluorescent channel #1 using image from FIG. 24A, and to enumerate cells of interest in fluorescent channel #2, using image from FIG. 24B. This function is used to calculate percent-positive cells of interest—here, 58% PR+ cells. (B) Microsphere calibrants evaluated for signal intensity using point-and-click software tool. In the example shown here, ‘LO’ intensity=34 and ‘HI’ intensity=176 on the 256-point intensity scale. (C) Left, data table showing single-cell intensity data for PR+ cells shown in fluorescent channel #2 image above. Right, data table information displayed as histogram, with microsphere calibrant values also shown. Mean cell population intensity=45/256, closer to ‘LO’ intensity. This information, in addition to percent-positive, can be used to score patient tissue according to accepted pathological scoring methods.

### DETAILED DESCRIPTION OF THE INVENTION

### High-Affinity Immunopolymers

The instant disclosure provides in one aspect a high affinity immunopolymer comprising a plurality of antibodies, a plurality of coupling proteins, and a plurality of detectable labels, wherein the plurality of antibodies and the plurality of coupling proteins are associated by a high-efficiency conjugation moiety. The high affinity of the immunopolymer is believed to be achieved, at least in part, due to the presence of multiple antibodies in a single construct. The multiple antibodies result in multiple binding events to multiple proximal antigens and thus high affinity. The association of the antibodies and the coupling proteins in the immunopolymer is achieved with high efficiency and a high degree of specificity at low concentrations through the high-efficiency conjugation moiety.

As just mentioned, the instant immunopolymers comprise a plurality of antibodies. As is well known in the art, antibodies are glycoproteins belonging to the immunoglobulin superfamily. Antibodies typically comprise two large heavy chains and two small light chains, but various alternative or modified antibody structures may be suitably employed in the conjugates of the instant disclosure.

For example, the antibodies may comprise natural antibodies, artificial antibodies, genetically engineered antibodies, monovalent antibodies, polyvalent antibodies, monoclonal antibodies, polyclonal antibodies, camelids, monobodies, single-chain variable fragments (scFvs) and/or fragments or derivatives thereof, including Fab fragments and F(ab′)₂ fragments. In certain applications, the antibody or immunoglobulin molecules may be monoclonal, polyclonal, monospecific, polyspecific, humanized, single-chain, chimeric, camelid single domain, shark single domain, synthetic, recombinant, hybrid, mutated, CDR-grafted antibodies, and/or fragments or derivatives thereof. In certain embodiments, antibodies may be derived from mammalian species, for example, human, rat, mouse, goat, guinea pig, donkey, rabbit, horse, llama, or camel. In other embodiments, antibodies may be derived from avian species, such as chicken or duck. The origin of the antibody is defined by the genomic sequence, irrespective of the method of production. The antibodies may be of various isotypes, e.g., IgG, IgM, IgA, IgD, IgE or subclasses, e.g., IgG1, IgG2, IgG3, IgG4. The antibodies may be produced recombinantly, or by other means, which may include antibody fragments that are still capable of binding an antigen, for example, an Fab, an F(ab)₂, Fv, scFv, VhH, and/or V-NAR. The antibodies, including antibody fragments, may be recombinantly engineered to include an epitope, for example, a peptide. In certain embodiments, the epitope may be a Myc tag, a FLAG tag, an HA tag, an S tag, a Streptag, an His tag, a V5 tag. In certain embodiments, the peptide tag may serve as a FlAsh tag, a biotinylation tag, Sfp tag, or other peptide subject to covalent modification.

The antibodies of the instant immunopolymers may be chemically modified to include a hapten, for example a small molecule or a peptide. The hapten may be a nitrophenyl, a dinitrophenyl, a digoxygenin, a biotin, a Myc tag, a FLAG tag, an HA tag, an S tag, a Streptag, a His tag, a V5 tag, a ReAsh tag, a FlAsh tag, a biotinylation tag, Sfp tag, or other chemical or peptide tag subject to covalent modification. Inclusion of an epitope or hapten in an antibody or antibody fragment may facilitate subsequent binding of a molecular probe, detectable component, binding moiety, or signal generating moiety. In certain embodiments, peptide tag haptens chemically conjugated to protein binders may be used in conjunction with anti-peptide tag antibody-signal detector conjugates in singleplex and multiplex immunodetection assays. The antibodies may comprise, for example, hybrid antibodies having at least two antigen or epitope binding sites, single polypeptide chain antibodies, bispecific recombinant antibodies (e.g. quadromes, triomes), interspecies hybrid antibodies, and molecules that have been chemically modified and may be regarded as derivatives of such molecules.

Suitable polyclonal antibodies for use in the instant immunopolymers may be produced through a variety of methods. For example, various animals may be immunized for this purpose by injecting them with an antigen, for example the target biological molecule, or another molecule sharing an epitope of the target biological molecule. Such antigen molecules may be of natural origin or obtained by DNA recombination or synthetic methods, or fragments thereof, and the desired polyclonal antibodies may be obtained from the resulting sera and may be purified. Alternatively, intact cells that array the target biological molecule may be used. Various adjuvants may also be used for increasing the immune response to the administration of antigen, depending on the animal selected for immunization. Examples of these adjuvants include Freund's adjuvant, mineral gels such as aluminum hydroxide, surfactant substances such as polyanions, peptides, oil emulsions, haemocyanins, dinitrophenol, or lysolecithin.

Suitable monoclonal antibodies for use in the instant immunopolymers are typically obtained from hybridoma cells, which are prepared by the fusion of spleen cells from a mouse that has been immunized with the desired antigen and myeloma cells. Cells expressing the desired antibody are then identified by their ability to bind the desired antigen. Stable hybridoma clones that produce significant amounts of the desired antibody may then be cultured to generate the antibody in useful amounts.

Accordingly, in some embodiments, the antibodies of the instant immunopolymers are primary antibodies, secondary antibodies, or anti-hapten antibodies. More specifically, a primary antibody may be an anti-estrogen receptor antibody, an anti-Her2 antibody, an anti-progesterone receptor antibody, an anti-EGFR antibody, an anti-cytokeratin antibody, an anti-Ki67 antibody, an anti-p53 antibody, or an antibody capable of binding any other such target antigen.

In other specific embodiments, the secondary antibody may be an anti-rabbit antibody, an anti-mouse antibody, an anti-goat antibody, an anti-rat antibody, an anti-human antibody, or any other such secondary antibody.

In still other specific embodiments, the anti-hapten antibody may be an anti-dinitrophenyl (DNP) antibody, an anti-digoxigenin (DIG) antibody, an anti-biotin (MO) antibody, an anti-trinitrophenyl (TNP) antibody, an anti-5-bromodeoxyuridine (BrdU) antibody, an anti-3-nitrotyrosine (3-NT) antibody, or an anti-drug antibody, although any other suitable anti-hapten antibody may be utilized in the instant immunopolymers.

As also noted above, the instant immunopolymers also comprise a plurality of coupling proteins. The coupling proteins in some of the instant immunopolymer embodiments serve to connect the plurality of antibodies to one another and thus to provide for multiple antibody binding sites. The coupling proteins usefully employed in the instant immunopolymers may be any suitable protein, so long as the protein has a plurality of selectively-reactive residues available for linking with the antibody. In addition, the linking of the coupling protein and the antibody should not significantly impair the interaction of the antibody with its target antigen. It is also desirable that the coupling protein not be otherwise reactive and not itself have interfering absorbance or fluorescence, so as to avoid any background signal from the protein itself. Ideally, such coupling proteins should also be readily available at high purity and low cost.

It should be understood that the choice of coupling protein may allow for the precise control of the linking reaction, including, for example, the size of the resulting immunopolymer, the extent of crosslinking within the immunopolymer, the spacing between antibodies within the immunopolymer, and so forth. For example, the number of reactive groups on the surface of a coupling protein will affect the extent of modification by the conjugating reagent and thus the degree of crosslinking by the high-efficiency conjugation moiety. Reactive thiol groups are relatively uncommon on the surface of soluble proteins, so the use of thiol-reactive conjugating reagents will typically result a relatively lower level of protein modification, a limited extent of crosslinking, and a relatively small size of the resulting immunopolymer. Reactive amino groups are much more common on the surface of soluble proteins, and the use of amine-reactive conjugating reagents will therefore typically result in a relatively higher level of protein modification, a larger extent of crosslinking, and a relatively larger size of the resulting immunopolymer.

The choice of large or small coupling proteins can likewise be used to modulate the features of the resulting immunopolymers, as can the extent of reaction with the relevant conjugating reagent. For example, large coupling proteins may provide larger spacing between attached antibodies and thus allow for improved efficiency in the binding of the antibodies of the immunopolymers to their antigens. Small coupling proteins may limit the extent of crosslinking and the size of the immunopolymers and thus provide advantages in some circumstances.

Additionally, if the coupling protein has a large number of reactive amino groups or other reactive groups, the extent of modification of the reactive groups by the conjugating reagent may be titrated as desired, for example by using a limited amount of the conjugating reagent relative to the number of reactive groups on the coupling protein. Similar titration of reactive groups on the antibody component of the immunopolymer may likewise be possible. Any remaining unreacted groups on the coupling protein or on the antibody may, if desired, be used for one or more subsequent modification reactions, either with an additional conjugating reagent or reagents, or with one or more reactive detectable labels.

Examples of the variation in the use of coupling proteins to modulate the linkages with antibodies and thus the properties of the immunopolymers are further described below. Additional variation is within the skill of those in the art. Indeed, the ability to modulate the properties of the inventive high-affinity immunopolymers by varying the modification of the coupling proteins, the modification of the antibodies, and the association of the antibodies and the coupling proteins by the high-efficiency linkage is one of the significant advantages of the instant immunopolymers.

Examples of suitable coupling proteins include albumins and immunoglobulins, although many other proteins could serve this purpose, as would be understood by those of ordinary skill in the art. In specific embodiments, the albumin is a serum albumin, such as, for example, bovine serum albumin (BSA). In other embodiments the albumin is ovalbumin. In still other specific embodiments, the immunoglobulin is an immunoglobulin G, an immunoglobulin A, an immunoglobulin M, or is a camelid immunoglobulin. It should be understood that immunoglobulins used as coupling proteins in the instant immunopolymers are not necessarily capable of binding to the antigen targets of the immunopolymers. The antigen-targeting functionality of an immunopolymer is provided by the antibody component of the immunopolymer.

The high-affinity immunopolymers of the instant disclosure further comprise a plurality of detectable labels. Such labels facilitate the identification and quantitation of the antigens bound by the immunopolymers in the various immunoassays described herein and in other suitable immunoassays. The detectable labels should be capable of suitable attachment to the antibodies, to the coupling proteins, or to both. In addition, attachment of the detectable labels to the immunopolymers should occur without significantly impairing the interaction of the antibody component of the immunopolymers with their target antigens.

In some embodiments, the detectable label may be directly detectable, such that it may be detected without the need for any additional components. For example, a directly detectable label may be a fluorescent dye, a biofluorescent protein, such as, for example, a phycoerythrin, an allophycocyanin, a peridinin chlorophyll protein complex (“PerCP”), a green fluorescent protein (“GFP”) or a derivative thereof (for example, a red fluorescent protein, a cyan fluorescent protein, or a blue fluorescent protein), luciferase (e.g., firefly luciferase, renilla luciferase, genetically modified luciferase, or click beetle luciferase), or coral-derived cyan and red fluorescent proteins (as well as variants of the red fluorescent protein derived from coral, such as the yellow, orange, and far-red variants), a luminescent species, including a chemiluminescent species, an electrochemiluminescent species, or a bioluminescent species, a phosphorescent species, a radioactive substance, a nanoparticle, a SERS nanoparticle, a quantum dot or other fluorescent crystalline nanoparticle, a diffracting particle, a Raman particle, a metal particle, including a chelated metal, a magnetic particle, a microsphere, an RFID tag, a microbarcode particle, or a combination of these labels.

In certain embodiments, the detectable label may be indirectly detectable, such that it may require the employment of one or more additional components for detection. For example, an indirectly detectable label may be an enzyme that effects a color change in a suitable substrate, as well as other molecules that may be specifically recognized by another substance carrying a label or that may react with a substance carrying a label. Non-limiting examples of suitable indirectly detectable labels include enzymes such as a peroxidase, an alkaline phosphatase, a glucose oxidase, and the like. In specific embodiments, the peroxidase is a horseradish peroxidase or a soybean peroxidase. Other examples of indirectly detectable labels include haptens such as, for example, a small molecule or a peptide. Non-limiting exemplary haptens include nitrophenyl, dinitrophenyl, digoxygenin, biotin, a Myc tag, a FLAG tag, an HA tag, an S tag, a Streptag, a His tag, a V5 tag, a ReAsh tag, a FlAsh tag, a biotinylation tag, an Sfp tag, or another chemical or peptide tag subject to covalent modification.

In some embodiments, the detectable labels are linked either to the plurality of antibodies or to the plurality of coupling proteins. In specific embodiments, the detectable labels are linked to the plurality of antibodies. In other specific embodiments, the detectable labels are linked to the plurality of coupling proteins. In still other specific embodiments, the detectable labels are linked both to the plurality of antibodies and to the plurality of coupling proteins. It should be understood that the specific attachment point and means of attachment of the detectable label to the immunopolymer is not believed to be critical, so long as the attachment does not significantly reduce the detectability of the label or significantly impair the ability of the immunopolymer to bind to its target antigen.

In preferred embodiments, the detectable label is a fluorescent dye. Non-limiting examples of suitable fluorescent dyes may be found in the catalogues of Life Technologies/Molecular Probes (Eugene, Oreg.) and Thermo Scientific Pierce Protein Research Products (Rockford, Ill.), which are incorporated by reference herein in their entireties. Exemplary dyes include fluorescein, rhodamine, and other xanthene dye derivatives, cyanine dyes and their derivatives, naphthalene dyes and their derivatives, coumarin dyes and their derivatives, oxadiazole dyes and their derivatives, anthracene dyes and their derivatives, pyrene dyes and their derivatives, and BODIPY dyes and their derivatives. Preferred fluorescent dyes include the DyLight fluorophore family, available from Thermo Scientific Pierce Protein Research Products.

As described in further detail below, the plurality of antibodies and the plurality of coupling proteins are associated with one another in the high-affinity immunopolymers by a high-efficiency conjugation moiety. Because the immunopolymers are preferably synthesized with relatively low molar concentrations of starting materials, and because those starting materials, for example antigen-specific antibodies, are expensive and are available in relatively small chemical quantities, it is highly desirable that formation of the conjugation moiety be as efficient and specific as possible and that it forms completely, or nearly completely, at low molar concentrations of reactants. Specifically, it is desirable that the conjugation moiety be capable of associating the antibodies and coupling proteins with rapid kinetics and/or high association constants and that the association reaction therefore be as efficient as possible in terms of its completion.

The high-efficiency conjugation moieties of the instant immunopolymers are typically formed, as described in more detail below, by separate modification of each component of the immunopolymer with complementary conjugating reagents. The complementary conjugating reagents additionally include a further reactive moiety, for example a thiol-reactive or an amino-reactive moiety, that allows the conjugating reagents to be attached to the relevant immunopolymer component, for example to the antibodies and to the coupling proteins. After the antibodies and the coupling proteins have been modified by the respective complementary conjugating reagents, typically at multiple locations on each entity, the complementary conjugating features on the modified components associate with one another in a highly efficient and specific manner to form the conjugation moiety.

Depending on the situation, the high-efficiency conjugation moiety of the instant immunopolymers may be a covalent or non-covalent conjugation moiety. In specific embodiments, the high-efficiency conjugation moiety is a covalent conjugation moiety, for example, a hydrazone, an oxime, or another suitable Schiff base moiety. Non-limiting examples of such conjugation moieties may be found, for example, in U.S. Pat. No. 7,102,024, which is incorporated by reference herein in its entirety for all purposes. These conjugation moieties may be formed by reaction of a primary amino group on the conjugating reagent attached to one component of the immunopolymer with a complementary carbonyl group on the conjugating reagent attached to the other component of the immunopolymer.

For example, hydrazone conjugation moieties may be formed by the reaction of a hydrazino group, or a protected hydrazino group, with a carbonyl moiety. Exemplary hydrazino groups include aliphatic, aromatic, or heteroaromatic hydrazine, semicarbazide, carbazide, hydrazide, thiosemicarbazide, thiocarbazide, carbonic acid dihydrazine, or hydrazine carboxylate groups. See U.S. Pat. No. 7,102,024. Oxime conjugation moieties may be formed by the reaction of an oxyamino group, or a protected oxyamino group, with a carbonyl moiety. Exemplary oxyamino groups are described below. The hydrazino and oxyamino groups may be protected by formation of a salt of the hydrazino or oxyamino group, including but not limited to, mineral acid salts, such as but not limited to hydrochlorides and sulfates, and salts of organic acids, such as but not limited to acetates, lactates, malates, tartrates, citrates, ascorbates, succinates, butyrates, valerates and fumarates, or any amino or hydrazino protecting group known to those of skill in the art (see, e.g., Greene et al. (1999) Protective Groups in Organic Synthesis (3rd Ed.) (J. Wiley Sons, Inc.)). The carbonyl moiety used to generate a Schiff base conjugation moiety is any carbonyl-containing group capable of forming a hydrazone or oxime linkage with one or more of the above hydrazino or oxyamino moieties. Preferred carbonyl moieties include aldehydes and ketones, in particular aromatic aldehydes and ketones. In preferred embodiments of the instant disclosure, the high-efficiency conjugation moiety is formed by the reaction of an oxyamino-containing component and an aromatic aldehyde-containing component.

The high-efficiency conjugation moiety of the instant immunopolymers may alternatively be formed by a “click” reaction, for example the copper-catalyzed reaction of an azide-substituted component with an alkyne-substituted component to form a triazole conjugation moiety. See Kolb et al. (2001) Angew. Chem. Int. Ed. Engl. 40:2004; Evans (2007) Aus. J. Chem. 60:384. Copper-free variants of this reaction, for example the strain-promoted azide-alkyne click reaction, may also be used to form the high-efficiency conjugation moiety. See, e.g., Baskin et al. (2007) Proc. Natl. Acad. Sci. U.S.A. 104:16793-97. Other click reaction variants include the reaction of a tetrazine-substituted component with either an isonitrile-substituted component (Stockmann et al. (2011) Org. Biomol. Chem. 9:7303) or a strained alkene-substituted component (Karver et al. (2011) Bioconjugate Chem. 22:2263).

The basic features of a click reaction are well understood by those of ordinary skill in the art. See Kolb et al. (2001) Angew. Chem. Int. Ed. Engl. 40:2004. Useful click reactions include generally but are not limited to [3+2] cycloadditions, such as the Huisgen 1,3-dipolar cycloaddition, and in particular the Cu(I)-catalyzed stepwise variant, thiol-ene click reactions, Diels-Alder reactions and inverse electron demand Diels-Alder reactions, [4+1] cycloadditions between isonitriles (isocyanides) and tetrazines, nucleophilic substitutions, especially to small strained rings like epoxy and aziridine compounds, carbonyl-chemistry-like formation of ureas, and some addition reactions to carbon-carbon double bonds. Any of the above reactions may be used without limitation to generate a covalent high-efficiency conjugation moiety in the instant immunopolymers.

In other embodiments, the high-efficiency conjugation moiety is a non-covalent conjugation moiety. Non-limiting examples of a non-covalent conjugation moiety include an oligonucleotide hybridization pair or a protein-ligand binding pair. In specific embodiments, the protein-ligand binding pair is an avidin-biotin pair, a streptavidin-biotin pair, or another protein-biotin binding pair (see generally Avidin-Biotin Technology, Meth. Enzymol. (1990) volume 184, Academic Press; Avidin-Biotin Interactions: Methods and Applications (2008) McMahon, ed., Humana; Molecular Probes® Handbook, Chapter 4 (2010)), an antibody-hapten binding pair (see generally Molecular Probes® Handbook, Chapter 4 (2010)), an S-peptide tag-S-protein binding pair (Kim and Raines (1993) Protein Sci. 2:348-56), or any other high-affinity peptide-peptide or peptide-protein binding pair. Such high-affinity non-covalent conjugation moieties are well known in the art. Reactive versions of the respective conjugating pairs, for example thiol-reactive or amino-reactive versions, are also well known in the art. These conjugating reagents may be used to modify the respective antibodies and the coupling proteins, typically at multiple locations on each entity. The antibodies and coupling proteins may then be mixed in order to allow the complementary features, for example the oligonucleotide hybridization pair or the protein-ligand binding pair, to associate with one another and form a non-covalent high-efficiency conjugation moiety. All of the above-described covalent and non-covalent linking groups are capable of highly efficient association reactions and are thus well suited for use in generation of the instant immunopolymers.

In some embodiments, the high-efficiency conjugation moiety is at least 50%, 80%, 90%, 93%, 95%, 97%, 98%, 99%, or even more efficient in coupling the antibodies and the coupling proteins. In more specific embodiments, the high-efficiency conjugation moiety is at least 50%, 80%, 90%, 93%, 95%, 97%, 98%, 99%, or even more efficient at a protein concentration of no more than 0.5 mg/mL. In some embodiments, the efficiencies are achieved at no more than 0.5 mg/mL, no more than 0.2 mg/mL, no more than 0.1 mg/mL, no more than 0.05 mg/mL, no more than 0.02 mg/mL, no more than 0.01 mg/mL, or even lower protein concentrations. Since coupling proteins are typically used in excess of antibodies in the preparation of the instant immunopolymers, the efficiency of a linking reaction is typically judged by the extent of conversion of the antibody component of the association reaction to immunopolymer product. For example, a high-efficiency conjugation moiety that is at least 50% efficient in coupling the antibodies and the coupling proteins is a moiety that results in at least 50% of the starting antibody being converted to an immunopolymer in the association reaction.

As noted above, one of the advantages of the instant immunopolymers is the presence of multiple antibodies within a single immunopolymer and thus the increased avidity of antigen binding sites in the immunopolymer. In some embodiments, an immunopolymer of the instant disclosure comprises at least three antibodies. In more specific embodiments, the immunopolymer comprises at least 4, at least 6, at least 8, at least 12, at least 16, at least 20, or even more antibodies. Furthermore, in some embodiments, an immunopolymer of the instant disclosure comprises at least three coupling proteins, and in more specific embodiments, the immunopolymer comprises at least 4, at least 6, at least 8, at least 12, at least 16, at least 20, or even more coupling proteins. It should also be understood that, although the relationship may not always follow, the immunopolymers typically contain a similar number of antibodies and coupling proteins. Accordingly, in some embodiments, the instant immunopolymers comprise at least three antibodies and at least three coupling proteins. In specific embodiments, the immunopolymers comprise at least 4 antibodies and at least 4 coupling proteins, at least 6 antibodies and at least 6 coupling proteins, at least 8 antibodies and at least 8 coupling proteins, at least 12 antibodies and at least 12 coupling proteins, at least 16 antibodies and at least 16 coupling proteins, at least 20 antibodies and at least 20 coupling proteins, or even more antibodies and coupling proteins.

The linking of antibodies and coupling proteins in the instant immunopolymers results in the formation of large complexes. Accordingly, in some embodiments, the immunopolymer has an average molecular weight of at least 200K, 250K, 300K, 400K, 600K, 800K, 1,000K, or even higher. Furthermore, the linking of antibodies and coupling proteins, each of which may comprise one or more detectable labels results in immunopolymers containing a large number of detectable labels. Such immunopolymers are thus readily detectable at extremely low concentrations. In embodiments, the immunopolymer of the disclosure comprises an average of at least 4, 8, 12, 16, 20, 24, 28, 32, or even more detectable labels.

### Methods of Detection

In another aspect, the instant disclosure provides methods of detecting an antigen, comprising reacting a first antigen with any of the high-affinity immunopolymers described above, and detecting the high-affinity immunopolymer.

In embodiments, the method of detection is an immunohistochemical method. As described above, immunohistochemical staining is widely used technique that is applied frequently to the diagnosis of abnormal cells, such as tumor cells. Specific molecular markers are characteristic of a particular tumor cell, for example a breast cancer cell. IHC is also frequently used to understand the distribution and localization of biomarkers and differentially expressed proteins in different parts of a biological tissue.

In specific embodiments, the first antigen detected by the method is present within a tissue section. Detection of antigens within tissue sections is well understood by those of skill in the clinical pathology arts. Exemplary methods of detecting antigens within a tissue section are provided, for example, in Immunohistochemical Staining Methods, 6^{th} ed. (Dako/Agilent Technologies).

In other specific embodiments, the first antigen detected by the method is in or on a cell. Such detection is well understood, for example, by those of skill in the art of cytometry. In some embodiments, the first antigen may be on the surface of a cell. In other embodiments, the first antigen may be in the cytoplasm of a cell. In still other embodiments, the first antigen may be in the nucleus of a cell. In some embodiments, the first antigen may be in more than one location in the cell.

The tissue analyzed according to the above methods may be any suitable tissue sample. For example, in some embodiments, the tissue may be connective tissue, muscle tissue, nervous tissue, or epithelial tissue. Likewise, the tissue analyzed may be obtained from any organ of interest. Non-limiting examples of suitable tissues include breast, colon, ovary, skin, pancreas, prostate, liver, kidney, heart, lymphatic system, stomach, brain, lung, and blood.

In some embodiments, the detecting step is a fluorescence detection step. Suitable fluorescence detection labels are described in detail above.

In some embodiments, the method of detection further comprises the step of sorting cells that have bound the first immunopolymer. Cell sorting is a well understood technique within the art of flow cytometry. Exemplary flow cytometry methods of detection are provided, for example, in Practical Flow Cytometry, 4^{th} ed., Shapiro, Wiley-Liss, 2003; Handbook of Flow Cytometry Methods, Robinson, ed., Wiley-Liss, 1993; and Flow Cytometry in Clinical Diagnosis, 4^{th} ed., Carey et al., eds, ASCP Press, 2007. The use of hydrazone-linked antibody-oligonucleotide conjugates in quantitative multiplexed immunoassays, in particular, in quantitative flow cytometric assays, is described in PCT International Publication No. WO 2013/188756 and in Flor et al. (2013) Chembiochem. 15:267-75.

In some embodiments, the method of detection further comprises reacting a second antigen with a second high-affinity immunopolymer, thus allowing for multiplexing of the immunoassay. The second high-affinity immunopolymer may be any of the immunopolymers described in detail above. In more specific embodiments, the method of detection may further comprise reacting a third antigen, a fourth antigen, a fifth antigen, or even more antigens with additional immunopolymers of the invention. As described in detail above, the ability of the instant immunopolymers to be used in such higher-level multiplexed immunoassays is a major advantage of these reagents.

The instant immunopolymers may likewise be usefully employed in a variety of other immunochemical methods of detection, including without limitation microscopic imaging, pretargeting imaging, and other types of in vivo tumor and tissue imaging, high content screening (HCS), immunocytochemistry (ICC), immunomagnetic cellular depletion, immunomagnetic cell capture, in situ hybridization (ISH), fluorescent in situ hybridization (FISH), sandwich assays, general affinity assays, enzyme immuno-as say (EIA), enzyme linked immuno-assay (ELISA), ELISpot, mass cytometry (CyTOF), arrays including microsphere arrays, multiplex microsphere array, microarray, antibody array, cellular array, solution phase capture, lateral flow assays, chemiluminescence detection, infrared detection, blotting methods, including Western blots, Southwestern blot, dot blot, tissue blot, and the like, or combinations thereof.

The antigens recognized by the antibodies of the instant immunopolymers may be either polypeptide antigens, such as, for example, cellular proteins of interest or other antibodies, or small-molecule antigens, such as haptens. Other antigens may also be usefully targeted by the instant immunopolymers, as would be understood by those of ordinary skill in the art. For example, targets of the instant immunopolymers include proteins, microorganisms, viruses, bacteria, drugs, hormones, toxins, biomolecules, lipids, carbohydrates, nucleic acids, synthetic molecules, modified proteins, and the like.

The above methods find use in research and clinical settings, without limitation. They may be used for diagnostic purposes, including predictive screening and in other types of prognostic assays, for example in a diagnostic laboratory setting or for point of care testing.

### Methods of Preparation

In another aspect, the instant disclosure provides novel methods of preparing high-affinity immunopolymers such as the immunopolymers described above. In embodiments, the methods comprise the steps of modifying an antibody with a first conjugating reagent, modifying a coupling protein with a second conjugating reagent, and reacting the modified antibody with the modified coupling protein to generate the high-affinity immunopolymer. In these reactions, the first linking reagent and the second linking reagent associate with one another at high efficiency.

By high-efficiency, it is meant that the efficiency of conversion of modified antibody to immunopolymer is at least 50%, 70%, 90%, 95%, or 99% complete under the conditions of the conjugation reaction. In some embodiments, these efficiencies are achieved at no more than 0.5 mg/mL, no more than 0.2 mg/mL, no more than 0.1 mg/mL, no more than 0.05 mg/mL, no more than 0.02 mg/mL, no more than 0.01 mg/mL, or even lower protein concentrations.

The antibodies and coupling proteins usefully employed in the methods of preparation include any of the antibodies and coupling proteins described above. The first and second conjugating reagents are chosen according to the desired outcomes. In particular, high-efficiency conjugating reagents capable of specific and selective reaction with amino or thiol groups are of particular utility in the modification of proteins, such as antibodies and coupling proteins. In addition, the first and second conjugating reagents are chosen for their ability to associate with one another at high efficiency, and thus to create the high-efficiency conjugation moiety of the above-described high-affinity immunopolymers.

As described above, the resulting conjugation moiety may be a covalent moiety or a non-covalent moiety, and the first and second conjugating reagents used to prepare the modified antibodies and modified coupling proteins are chosen accordingly. For example, in the case of a non-covalent conjugation moiety, the first conjugating reagent preferably comprises a selectively reactive group to attach the reagent to particular reactive residues of the antibody and a first component of the conjugation pair. Likewise, the second conjugating reagent preferably comprises a selectively reactive group to attach the reagent to particular reactive residues of the coupling protein and a second component of the conjugation pair. The first and second components of the conjugation pairs are able to associate with one another non-covalently at high efficiency and thus to generate the high-affinity immunopolymer.

As previously described, examples of non-covalent conjugation moieties include oligonucleotide hybridization pairs and protein-ligand binding pairs. In the case of an oligonucleotide hybridization pair, for example, the antibody would be reacted with a first conjugating reagent that comprises one member of the hybridization pair, and the coupling protein would be reacted with a second conjugating reagent that comprises the second member of the hybridization pair. The modified antibody and the modified coupling protein can thus be mixed with one another, and the association of the two members of the hybridization pair generates the high-efficiency conjugation moiety.

Likewise, when a protein-ligand binding pair is used to generate the non-covalent conjugation moiety of the high-affinity immunopolymer, the antibody is reacted with a first conjugating reagent that comprises one or the other of the protein-ligand pair, and the coupling protein is reacted with a second conjugating reagent that comprises the complementary member of the protein-ligand pair. The so-modified antibody and coupling protein are then mixed with one another to generate the high-efficiency conjugation moiety.

As was described in detail above, examples of high-efficiency covalent conjugation moieties include hydrazones, oximes, other Schiff bases, and the products of any of the various click reactions. Exemplary hydrazino, oxyamino, and carbonyl conjugating reagents for use in forming the high-efficiency conjugation moieties are illustrated in U.S. Pat. No. 7,102,024 and can be adapted for use in the instant reaction methods. As described therein, the hydrazine moiety may be an aliphatic, aromatic, or heteroaromatic hydrazine, semicarbazide, carbazide, hydrazide, thiosemicarbazide, thiocarbazide, carbonic acid dihydrazine, or hydrazine carboxylate. The carbonyl moiety may be any carbonyl-containing group capable of forming a hydrazine or oxime linkage with one or more of the above-described hydrazine or oxyamino moieties. Preferred carbonyl moieties include aldehydes and ketones. Activated versions of some of these reagents, for use as conjugating reagents in the instant methods, are available commercially, for example from Solulink, Inc. (San Diego, Calif.) and Jena Bioscience GmbH (Jena, Germany).

The incorporation of hydrazine, oxyamino, and carbonyl-based monomers into oligonucleotides for use in immobilization and other conjugation reactions is described in U.S. Pat. Nos. 6,686,461; 7,173,125; and 7,999,098. Hydrazine-based and carbonyl-based bifunctional crosslinking reagents for use in the conjugation and immobilization of biomolecules is described in U.S. Pat. No. 6,800,728. The use of high-efficiency bisaryl-hydrazone linkers to form oligonucleotide conjugates in various detection assays and other applications is described in PCT International Publication No. WO 2012/071428. Each of the above references is hereby incorporated by reference herein in its entirety.

In some embodiments, the immunopolymers of the instant disclosure are prepared using novel conjugating reagents and conditions. For example a thiol-reactive maleimido oxyamino (MOA) conjugating reagent useful in the preparation of high-affinity immunopolymers may be prepared as shown in Scheme 1 (see also Example 1, below):

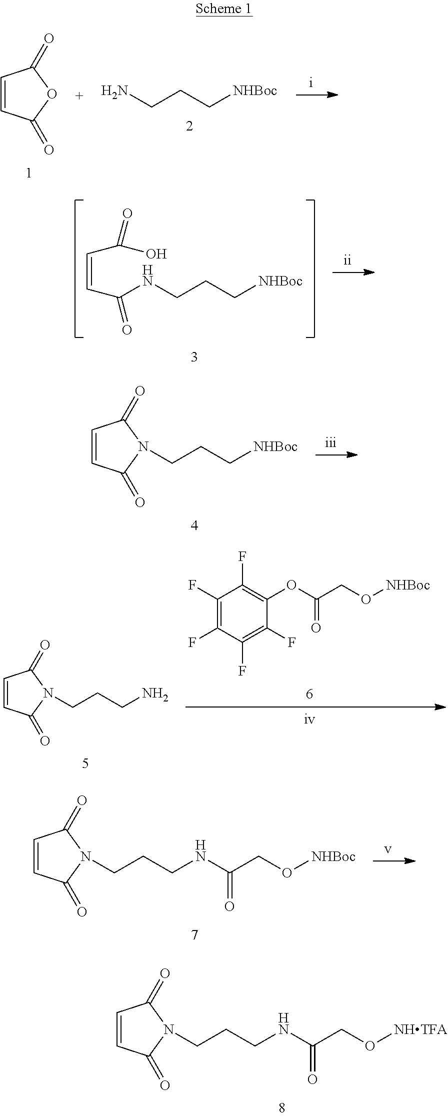

An amino-reactive oxyamino conjugating reagent (AOA) may be prepared as shown in Scheme 2 (see also Example 2, below):

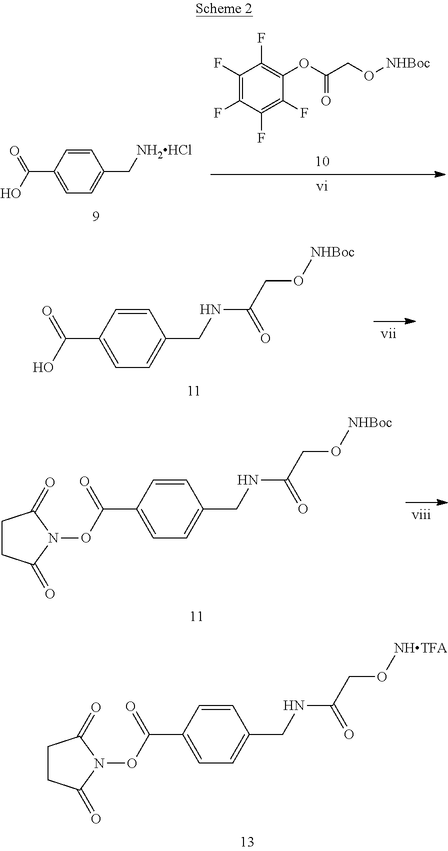

Alternative thiol-reactive and amino-reactive conjugating reagents may be prepared using variants of the above reaction schemes, as would be understood by those of ordinary skill in the art of synthetic chemistry. Such alternative reagents should be considered within the scope of the preparation methods disclosed herein.

Antibodies and coupling proteins modified using one or another of the above oxyamino-containing reagents may usefully be reacted with a complementary antibody or coupling protein that is itself modified with a carbonyl-containing reagent, for example, an aromatic aldehyde such as a formylbenzoate group. An example of such a conjugation reaction is shown in Scheme 3, where the R₁ and R₂ groups represent independently an antibody or a coupling protein.

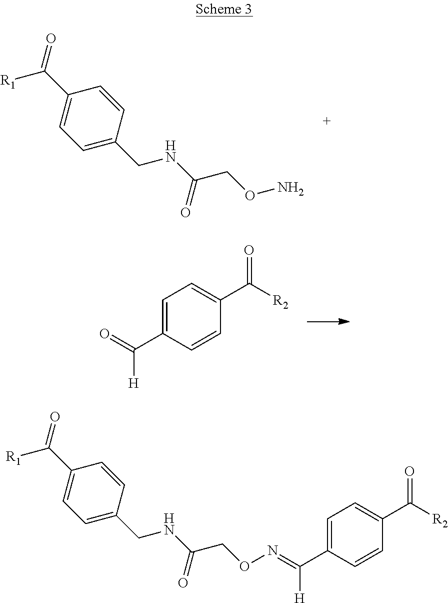

It should be understood that the relative orientation of the different members of the conjugation moiety-forming groups on the antibody and on the coupling protein are generally not believed to be important, so long as the groups are able to react with one another to form the high-efficiency conjugation moiety. In other words, in the example of Scheme 3, the R₁ group could be the antibody and the R₂ group could be the coupling protein, or the R₁ group could be the coupling protein and the R₂ group could be the antibody. The same is generally true for all of the above-described conjugating pairs, whether covalent or non-covalent.

The above-described polymerization methods provide several advantages over traditional polymerization methods, for example methods using bifunctional conjugation reagents. In particular, the reactions are specific, efficient, and stable. The specificity means that side reactions, such as homoconjugation reactions, do not occur, or occur at extremely low levels. The efficiency means that the reactions run to completion, or near completion, even at low protein concentrations, thus generating products in at or near stoichiometric amounts. The stability of the conjugation moieties formed means that the resultant immunopolymers can be used for a wide variety of purposes without concern that the conjugated products will dissociate during use. In some cases, the above polymerization methods allow the further advantage that the progress of the polymerization reaction may be monitored spectroscopically, since in some of the reactions a chromaphore is formed as the reaction occurs.

The synthesis and stabilities of hydrazone-linked adriamycin/monoclonal antibody conjugates are described in Kaneko et al. (1991) Bioconj. Chem. 2:133-41. The synthesis and protein-modifying properties of a series of aromatic hydrazides, hydrazines, and thiosemicarbazides are described in U.S. Pat. Nos. 5,206,370; 5,420,285; and 5,753,520. The generation of conjugationally-extended hydrazine compounds and fluorescent hydrazine compounds is described in U.S. Pat. No. 8,541,555.

### Reagent Mixtures and Methods of Calibration

In still another aspect, the disclosure provides reagent mixtures and methods of calibration for use in immunoassays. According to one embodiment, the reagent mixture comprises a first population and a second population of particles comprising a detectable agent, wherein the detectable agent is associated with the surface of the first population of particles at a first surface density and is associated with the surface of the second population of particles at a second surface density.

In specific embodiments, the particles of the reagent mixture are microspheres.

In some embodiments, the detectable agent of the reagent mixture is a cellular antigen, and in other embodiments, the detectable agent is a hapten.

In some embodiments, the reagent mixture further comprises a detectable immunoreagent specific for the agent. In certain embodiments, the detectable immunoreagent comprises a fluorescent label. It should be understood that the detectable agent may be detectable either directly or indirectly. For example, although a cellular antigen or a hapten may not itself emit any kind of detectable signal and therefore may not itself be directly detectable, it becomes detectable upon being bound by a detectable immunoreagent.

In preferred embodiments, the detectable immunoreagent is a high-affinity immunopolymer of the instant disclosure.

As described in further detail in Example 12, the reagent mixtures of the instant disclosure comprise at least two populations of calibrant particles, preferably microspheres, wherein the particles comprise a detectable agent that is present in each population of particles at a different surface density. If the same detectable agent is present in a sample of interest, for example in or on cells being analyzed by flow cytometry or in tissue sections being analyzed by immunohistochemistry, the particles can thus be used to calibrate the amount of the corresponding agent in the sample of interest. In specific embodiments, the reagent mixtures comprise at least two, at least three, at least four, at least six, or even more populations of calibrant particles, each population with a different surface density of the detectable agent.

In some embodiments, the detectable agent is a cellular antigen, although in preferred embodiments, the detectable agent is a hapten. The use of hapten-containing calibrant microspheres simplifies and increases the adaptability of the reagent mixture, because the calibrant mixture can be used in immunohistochemical assays for any cellular antigen, simply by using a haptenylated primary or secondary antibody that is specific for the desired target antigen. In these embodiments, the hapten, as the detectable agent, is detected using one or more of the high-affinity immunopolymers of the instant disclosure. A limited number detectable immunopolymers can thus be used to assay a wide variety of target antigens.

Exemplary haptens for use in the haptenylated calibrant particles of the instant reagent mixtures may be, for example, a nitrophenyl, a dinitrophenyl, a digoxygenin, a biotin, a Myc tag, a FLAG tag, an HA tag, an S tag, a Streptag, a His tag, a V5 tag, a ReAsh tag, a FlAsh tag, a biotinylation tag, Sfp tag, or other chemical or peptide tag.

In some embodiments, however, it may be desirable to use calibrant particles with a surface-accessible cellular antigen as the detectable agent, rather than a hapten.

The calibrant particles of the instant reagent mixtures may be prepared using any appropriate chemical or biochemical synthetic techniques. For example, a detectable agent, such as a hapten or a cellular antigen, may be reacted with activated or otherwise reactive particles, using controlled amounts of the hapten or cellular antigen in order to generate the different populations of particles with different densities of detectable agent, as would be understood by the skilled artisan. Suitable activated or otherwise reactive particles, for example carboxy-modified microspheres, are commercially available. Such particles may, for example, be modified using the same high-efficiency linking reagents that are described above for use in preparing the high-affinity immunopolymers of the instant disclosure, although other suitable methods of preparation are possible, as would be understood by those of ordinary skill in the art.

The calibrant reagent mixtures disclosed herein are suitable for use in a variety of immunochemical assays, including without limitation, flow cytometry, immunocytochemistry, immunohistochemistry, microscopy, and any other type of imaging. For comparison, the use of microspheres or microspheres in quantitative oligonucleotide-mediated multiparametric flow cytometry assays is described in PCT International Publication No. WO 2013/188756 and in Flor et al. (2013) Chembiochem. 15:267-75. The use of antibody- or aptamer-linked microspheres or other microparticles for calibration in bright-field microscopy is described in U.S. Patent Application Publication No. 2014/0113385.

Also provided herein are methods for calibration of an immunochemical assay, the methods comprising treating a population of cells with a first population and a second population of particles comprising a detectable agent, wherein the detectable agent is associated with the surface of the first population of particles at a first surface density and is associated with the surface of the second population of particles at a second surface density.

The particles used in the methods of calibration are preferably the calibrant particles of the above-described reagent mixtures. In some method embodiments, the cells and the particles are treated with a detectable immunoreagent specific for the detectable agent. In specific embodiments, the immunoreagent comprises a fluorescent label. In more specific embodiments, the immunoreagent is a high-affinity immunopolymer of the instant disclosure.

In some embodiments of the instant methods of calibration, the cells and the particles are separately treated with the detectable immunoreagent. For example, in the case of an immunohistochemical assay, it may be advantageous to mix the detectable immunoreagent, for example a high-affinity immunopolymer of the instant disclosure, with the different populations of particles prior to the application of the particles to the tissue sample. The tissue sample itself may be advantageously treated with the detectable immunoreagent prior to application of the calibrant particles to the tissue sample. In other embodiments, however, the cells and the particles may advantageously be jointly treated with the detectable immunoreagent. For example, a tissue sample may be treated with the calibrant particles in the absence of the detectable immunoreagent, and the tissue sample may subsequently be treated with the immunoreagent.

Depending on the detectable agent used, there may be additional steps in the calibration methods. For example, when the detectable agent of the calibration method is a hapten, the cell population may be treated with a haptenylated primary antibody prior to treatment with the hapten-specific detectable immunoreagent. Additional or alternative steps involving treatments with secondary antibodies may likewise be included in the methods, as would be understood by those of ordinary skill in the immunochemical arts.

In some embodiments, the methods of calibration further include the step of detecting the amount of detectable agent in the cells and in the particles. In specific embodiments, the methods further include the step of scoring the level of detectable agent in the cells, based on the amount of detectable agent in the different populations of particles. In some embodiments, the scoring step is performed automatically.

### Diagnostic Kits

In another aspect, the instant disclosure provides diagnostic kits for use in immunochemical assays for diagnostic or research purposes. The diagnostic kits comprise one or more immunopolymers of the instant disclosure, together with instructions for use in an immunoassay. In some embodiments, the kits further comprise an antibody, for example a primary antibody or a secondary antibody, that is recognized by the immunopolymer of the kit. In more specific embodiments, the antibody is conjugated with one or more haptens. For kits containing a haptenylated antibody, the included immunopolymer will preferably comprise anti-hapten antibodies. Furthermore, it should be understood that the antibody included in the instant kits will typically be a primary antibody, so that the kit may be used in immunoassays for the detection of a cellular antigen, for example in a tissue sample or in a suspension of cells. Labeling of the antibody with a hapten allows the antibody to be detected by an anti-hapten immunopolymer or mixture of anti-hapten immunopolymers. In some situations, however, it may be useful for the kit to provide a secondary antibody, for example an anti-mouse antibody, an anti-rabbit antibody, or the like. In these kits, the secondary antibody may either be haptenylated, and thus detectable by an anti-hapten immunopolymer or mixture of anti-hapten immunopolymers, or may be directly recognized by an immunopolymer comprising antibodies raised against an antibody of that particular species.

In some embodiments, the kits may further comprise at least one population of particles comprising a detectable agent for use in calibration of an immunochemical assay. In specific embodiments, the particles are microspheres. In other specific embodiments, the detectable agent is associated with the surface of the particles at a defined surface density. In some embodiments, the detectable agent is a cellular antigen or a hapten.

In further embodiments, the kits may comprise further components such as, for example, solid supports such as microplates or glass slides that may or may not be surface-modified with haptens, cellular antigens, antibodies, or chemical linkers, to provide a support for biological samples and/or microsphere calibrants; buffers of various compositions to enable usage of the kit for staining cells or tissues; and cellular counterstains to enable visualization of sample morphology. Kits may be provided in various formats and include some or all of the above listed components, or may include additional components not listed here.

### Therapeutic Agents

In another aspect, the instant disclosure provides immunopolymers useful as therapeutic agents. As is well understood in the art, antibodies, including all of the modified antibodies and antibody variants described above, without limitation, may be used in immunotherapies to treat individuals suffering from a wide variety of illnesses. Non-limiting examples of such therapeutic targets include cardiovascular disease, rheumatoid arthritis and other autoimmune diseases, such as Crohn's disease, ulcerative colitis, and the like, Alzheimer's disease, and a variety of cancers.

Exemplary FDA-approved therapeutic monoclonal antibodies include: abciximab, adalimumab, alemtuzumab, basiliximab, belimumab, bevacizumab, brentuximab vedotin, canakinumab, cetuximab, certolizumab pegol, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumumab, ranibizumab, rituximab, tocilizumab (atlizumab), tositumomab, trastuzumab, pembrolizumab, and vedolizumab. Other examples include therapeutic monoclonal antibodies that are currently under review by the FDA and that may obtain approval.

The high-affinity immunopolymers of the instant disclosure may usefully incorporate any of the above therapeutic antibodies, or combinations thereof, for use in targeted immunotherapies. In addition, novel antibodies that bind to the target antigens of any of the above antibodies, or combinations thereof, may likewise be usefully incorporated into the immunopolymers of the instant disclosure and thus result in effective immunotherapeutics. Furthermore, novel antigen targets that are related to the diseases treated by any of the above immunotherapies, or any other targetable disease, may likewise be used to prepare antibodies usefully incorporated into the instant immunopolymers for use as therapeutic agents.

In addition, the immunopolymers of the instant disclosure may be further modified to generate novel therapeutic agents. For example, the immunopolymers may be modified by attachment of a biologically active agent, such as a cytotoxic payload (for use in cancer treatments) or other drug, to generate an antibody-drug conjugate (ADC), in this case an immunopolymer-drug conjugate. Several ADCs have now received market approval in the U.S. Analogous immunopolymer-drug conjugates may be developed using the instant immunopolymers, or their derivatives, as would be understood by those of ordinary skill in the art.

It will be readily apparent to one of ordinary skill in the relevant arts that other suitable modifications and adaptations to the methods and applications described herein may be made without departing from the scope of the invention or any embodiment thereof. Having now described the present invention in detail, the same will be more clearly understood by reference to the following Examples, which are included herewith for purposes of illustration only and are not intended to be limiting of the invention.

### EXAMPLES

### Materials and Methods

Buffers and desalting columns. Modification Buffer (100 mM phosphate, 150 mM NaCl, pH 7.4-7.6), conjugation buffer (100 mM phosphate, 150 mM NaCl, pH 6.0), aniline buffer (100 mM, phosphate, 150 mM NaCl, 100 mM aniline, pH 6.0), PBS (10 mM phosphate, 150 mM NaCl, pH 7.0). “Zeba” desalting columns were obtained from ThermoPierce (Rockford, Ill.).

Amino-reactive fluorescent dyes. Dy488-OSu, Dy550-OSu and Dy650-OSu were purchased from ThermoPierce (Rockford, Ill.).

Antibodies. Unlabeled, purified anti-Her2, anti-estrogen receptor (ER), and anti-progesterone receptor (PR) rabbit monoclonal antibodies were purchased from Epitomics, Inc (Fremont, Calif.); anti-Ki67 mouse monoclonal antibody was purchased from Biolegend (San Diego, Calif.); anti-CD3 mouse monoclonal antibody was obtained from University of Chicago Monoclonal Antibody Facility (Chicago, Ill.). Unlabeled, purified goat anti-mouse, goat anti-rabbit, and donkey anti-goat polyclonal antibodies were purchased from ImmunoReagents Inc. (Raleigh, N.C.). Conventional fluorophore-labeled secondary antibodies were purchased from Jackson ImmunoResearch (West Grove, Pa.) and ThermoPierce (Asheville, N.C.). Conventional HRP-labeled anti-rabbit secondary antibody was purchased from GE Life Sciences (Piscataway, N.J.). Sources for miscellaneous antibodies and other reagents are noted in the Examples below.

### Tissue Staining and Image Processing Protocols

Immunofluorescence staining and imaging: The following protocols were employed in the below-described immunofluorescence staining experiment of formalin-fixed paraffin-embedded (FFPE) tissue samples. Tissue slides were imaged using an automated tissue slide scanner (IC200 Imager, Vala Sciences, San Diego, Calif.). The images were processed using open-source ImageJ software (NIH, Bethesda, Md.).

Unless otherwise indicated, all breast cancer tissue was obtained from the University of Chicago Department of Pathology (Chicago, Ill.).

### Immunofluorescence Staining Protocol

1. Slides were heated at 75° C. for 15 min.
2. Tissue was deparaffinized by immersion in xylene (2×5 min), followed by rehydration in descending ethanols (100%, 2×2 min; 95%, 70%, 50% ethanol, 1 min each). Slides were then rinsed in distilled water for 2 min.
3. Antigen retrieval was accomplished by steaming tissue slides in 10 mM citric acid, pH 6.0, for 15 min.
4. Slides were cooled to room temperature for 15 min.
5. Slides were washed under running tap water for 5 min.
6. Circles were drawn around the tissue sections using a hydrophobic pen to reduce the staining volume needed per section.
7. Endogenous biotin in tissue was blocked using Biotin Blocking Kit (Dako, Carpenteria, Calif.). In brief, tissue was exposed to avidin solution for 10 min., rinsed once with PBS, and then exposed to biotin solution for 10 min. at room temperature.
8. Slides were rinsed with PBS.
9. Slides were incubated with serum-free protein block (Dako) for 20 min. at room temperature.
10. Primary antibody or hapten-conjugated primary antibody was diluted into antibody diluent (Dako) at desired concentrations and added to the tissue, which was incubated overnight at 4° C.
11. Slides were washed with PBS+0.1% Tween-20, 3×5 min.
12. To each tissue section was added the fluorescent immunopolymer at the desired concentration and incubated for 1 h at room temperature.
13. Slides were washed with PBS+0.1% Tween 20, 3×5 min.
14. To slides was added a mounting medium containing nuclear counterstain (“VectaShield+DAPI”, Vector Laboratories, Burlingame, Calif.), and then slides were coverslipped and imaged.

### Triplex Immunoflourescence Staining Protocol and Modification Example

10. To the slide was added a solution of Her2-DNP, ER-DIG, and PR-BIO at desired concentrations (generally, 3.0 μg each) in antibody diluent. Tissue slides were incubated overnight at 4° C.
12. A solution of [(anti-DNP-BSA)-Dy550], [(anti-DIG-BSA)-Dy650], and [(anti-BIO-BSA)-Dy488] at desired concentrations (generally, 2.5-5.0 μg/mL) in antibody diluent. Tissue slides were incubated at room temperature for 1 h.

Immunoperoxidase staining and imaging: The following protocols were employed in the below-described staining experiment of formalin-fixed paraffin-embedded (FFPE) tissue samples. Tissue slides were imaged using an automated tissue slide scanner (IC200 Imager, Vala Sciences, San Diego, Calif.). The images were processed using open-source ImageJ software (NIH, Bethesda, Md.). Unless otherwise indicated all breast cancer tissue samples were obtained from the University of Chicago Department of Pathology (Chicago, Ill.).

### Immunoperoxidase Staining and Imaging Protocol

1. Slides were heated at 75° C. for 15 min.
2. Tissue was deparaffinized by immersion in xylene (2×5 min), followed by rehydration in descending ethanols (100%, 2×2 mM; 95%, 70%, 50% ethanol, 1 min each). Slides were then rinsed in distilled water for 2 min.
3. Antigen retrieval was accomplished by steaming tissue slides in 10 mM citric acid, pH 6.0, for 15 mM.
4. Slides were cooled to room temperature for 15 min.
5. Slides were washed under running tap water for 5 min.
6. Circles were drawn around the tissue sections using a hydrophobic pen to reduce the staining volume needed per section.
7. Endogenous peroxidase activity was blocked with hydrogen peroxide 3% for 15 mM at room temperature.
8. Slides were washed under running tap water for 5 min.
9. Endogenous biotin in tissue was blocked using Biotin Blocking Kit (Dako). In brief, tissue was exposed to avidin solution for 10 mM, rinsed once with PBS, and then exposed to biotin solution for 10 mM at room temperature.
10. Slides were rinsed with PBS.
11. Slides were incubated with serum-free protein block (Dako) for 20 min at room temperature.
12. Primary antibody or hapten-conjugated primary antibody was diluted into antibody diluent (Dako) at desired concentrations and added to tissue, which was incubated overnight at 4° C.
13. Slides were washed with PBS +0.1% Tween-20, 3×5 min.
14. To each tissue section was added the peroxidase-based enyzmatic immunopolymer (e.g. [(anti-Rb-BSA)-poly(HRP)] at the desired concentration and incubated for 1 h at room temperature.
15. Slides were washed with PBS.
16. DAB reagent was added for 5 min to visualize the HRP staining.
17. Slides were washed with PBS 3×5 min.
18. Nuclei were counterstained using standard hematoxylin stain.
19. Tissue was dehydraded by graded ethanols (50%, 70%, 95%, 2 min each; 100% ethanol, 2×2 min) followed by clearing in xylene (2×5 min).
20. To slides was added a mounting medium (“Cytoseal”, Thermo, Asheville, N.C.), and then slides were coverslipped and imaged.

### Example 1: Synthesis of Thiol-Reactive Maleimido Oxyamino (MOA) Conjugating Reagent

A thiol-reactive conjugating reagent was synthesized according to Scheme 1 (see above) as follows.

Synthesis of 3: To a solution of 3-Boc-aminopropylamine (1.65 g; 10.1 mmol; Oakwood Chemical) and diisopropylethylamine (1.89 mL; 11.2 mmol; 1.1 equiv) in DMF (20 mL) was added dropwise a solution of maleic anhydride (1.0 g; 10.1 mmol) in DMF (5.0 mL). The reaction mixture was stirred at room temperature for 2 h. TLC (100% ethyl acetate; ninhydrin visualization) indicated complete conversion to product. To the reaction mixture was added NHS (1.17 g; 10.1 mmol) and DCC (2.09 g; 10.1 mmol) and stirred at room temperature for 15 h. The reaction mixture was concentrated to dryness and the residue was partitioned between ethyl acetate and saturated sodium bicarbonate solution. The organic phase was further extracted with sodium bicarbonate solution (2×20 mL). The combined bicarbonate solutions extracts was acidified to pH 3.0 and extracted with DCM (3×40 mL). The combined organic extracts were washed with brine, dried over anhydrous magnesium sulfate, filtered and concentrated to yield 3.1 g of a colorless oil. The product 3 was used in the next step without purification.

Synthesis of 4: To a solution of 3 (0.5 g; 1.35 mmol) in dichloromethane (15 mL) was added (Bu)4N⁺I⁻ (100 mg; 0.27 mmol; 0.2 mol equiv) and a solution of NaOH (0.6 g; 1.5 mmol; 1.1 mol equiv) in water (15 mL). The reaction mixture was stirred rapidly for 15 hours. The phases were separated and the aqueous phase was further extracted with DCM (2×25 mL). The combined organic extracts were washed with brine, dried over anhydrous magnesium sulfate, filtered and concentrated to yield 0.275 g of a colorless oil. The oil was filtered through a plug of silica gel eluting with hex/EA (1/1). The eluant was concentrated to dryness to give 205 mg of 4 as confirmed by PMR analysis.

Synthesis of 6: To a solution of Boc-aminooxyacetic acid (2.0 g; 10.5 mmol; EMD Chemicals) in DCM (25 mL) was added pentafluorophenol (1.75 mL; 9.94 mmol; 0.95 equiv; Oakwood Chemical) and EDC (2.21 g; 11.5 mmol; 1.1 equiv). The reaction mixture was stirred overnight at room temperature. The solvent was removed on the rotavap and the residue was partitioned between ethyl acetate (30 mL) and saturated sodium bicarbonate solution (30 mL). The phases were separated and the aqueous phase was further extracted with ethyl acetate (20 mL). The combined organic extracts were washed with brine, dried over anhydrous magnesium sulfate, filtered and concentrated to give 2.46 g of a white solid.

Synthesis of 5: To a solution of 4 (175 mg; 0.69 mmol) in DCM (5 mL) was added trifluoroacetic acid (3 mL) the reaction mixture was stirred at room temperature for 2 h and concentrated to dryness. The residue was co-evaporated three times with toluene (3×10 mL). The residue 5 was used in the next step directly.

Synthesis of 7: To a solution of 5 (170 mg; 0.63 mmol) and triethylamine (331 μL; 1.90 mmol; 3 equiv) in DMF (5 mL) was added a solution of 6 (227 g; 0.63 mmol) in DMF (2 mL). The basicity of the reaction mixture was checked by placing a 1 μL aliquot on a wetted pH stick. Further TEA was added to increase the pH to ˜8. The reaction mixture was stirred at room temperature for 4 h. TLC (100% ethyl acetate; ninhydrin visualization) indicated one major new product. The reaction mixture was concentrated to dryness and partitioned between ethyl acetate and sodium bicarbonate. The bicarbonate wash was further extracted with ethyl acetate. The combined organic extracts were washed with brine, dried over magnesium sulfate, filtered and concentrated to give 145 mg of a colorless oil. PMR spectrum was excellent for desired product.

Synthesis of MOA (8): To a solution of 7 (50 mg; 0.20 mmol) in DCM (2 mL) was added trifluoroacetic acid (1 mL) the reaction mixture was stirred at room temperature for 2 h and concentrated to dryness. The residue was co-evaporated three times with toluene (3×10 mL) to yield 53 mg of a colorless oil. PMR analysis of the residue was excellent for 8 as the desired product.

### Example 2: Synthesis of Amino-Reactive Oxyamino (AOA) Conjugating Reagent

4-(N-(Boc-aminooxyacetamido)methyl)-benzoic acid (11): 4-(aminomethyl)benzoic acid (9) (302 mg; 2.0 mmol) and pentafluorophenyl ester (10) (716 mg; 2 mmol) in 6 ml of dry DMF were stirred overnight at room temperature. The reaction mixture was diluted with brine (30 mL) and acidified with dilute HCl to pH 2-3. The product was extracted with EtOAc (3×25 mL). The product 5 was purified by silica column flash chromatography (gradient elution from hexanes-CHC13-EtOAc-iPrOH (3:3:3:1) to hexanes-CHCl₃-EtOAc-iPrOH (3:3:3:5) to yield desired product (340 mg; 55% yield).

Succinimidyl 4-(N-(Boc-aminooxyacetamido)methyl)-benzoate (12): To a solution of 11 (120 mg; 0.36 mmol) in DCM (2 mL) was added NHS (42 mg; 0.36 mmol) and EDC (76 mg; 0.4 mol). The reaction mixture was stirred at room temperature for 15 h. The reaction mixture was diluted with DCM and washed with saturated sodium bicarbonate solution and brine, dried over anhydrous magnesium sulfate, filtered and concentrated to yield 130 mg of a pale yellow solid. PMR spectrum was excellent for desired product.

Succinimidyl 4-(N-(aminooxyacetamido)methyl)-benzoate TFA salt (13) (NHS-AOA): To a solution of 12 (15 mg; mmol) in DCM (2 mL) was added trifluoroacetic acid (1 mL). The reaction mixture was stirred at room temperature for 1 h and concentrated on the rotavap. The residue was co-evaporated three times with toluene. PMR of an aliquot of the product was excellent for desired product 13.

### Example 3: Synthesis of [(BSA-Fluorophore)-IgG] Immunopolymer Using Amino-Reactive HyNic Conjugating Reagent

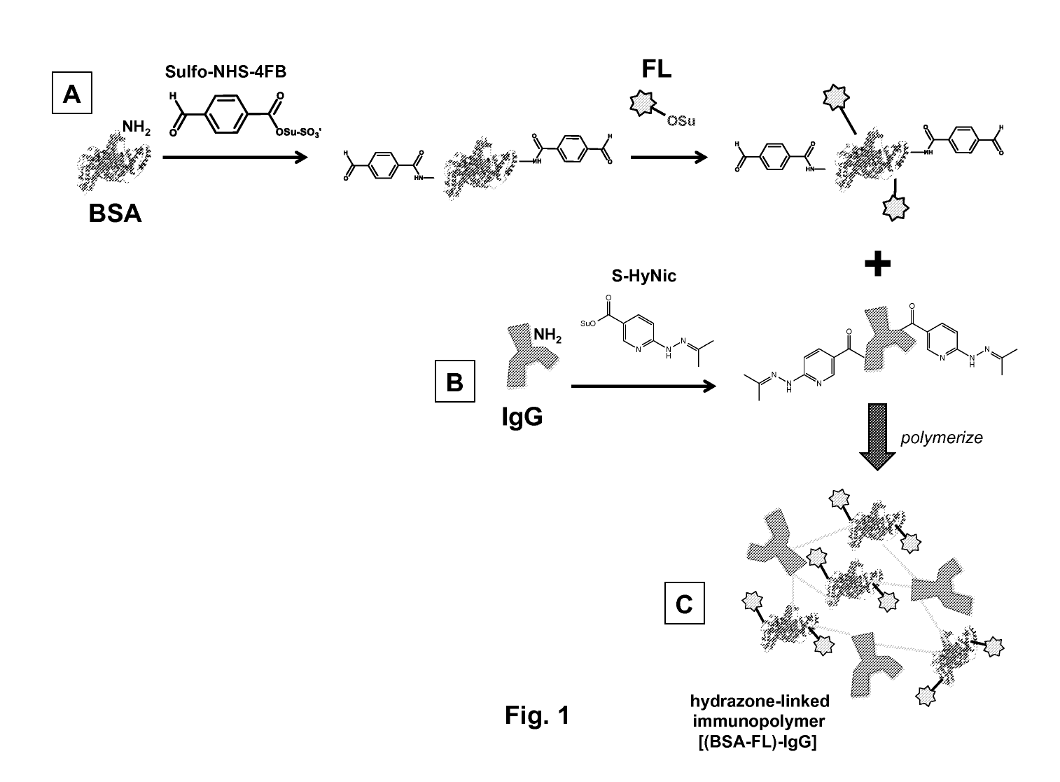

(BSA-fluorophore)-IgG immunopolymers were prepared using the following 4-step procedure, as illustrated schematically in FIG. 1. This strategy takes advantage of the HyNic/4FB conjugating reagent pair. See, e.g., U.S. Pat. No. 6,800,728. The steps include (1) modification of BSA with an amino-reactive 4-formylbenzoate (4FB), (2) modification of BSA-4FB with an amino-reactive fluorophore, (3) modification of antibody with succinimidyl 6-hydrazinonicotinate acetone hydrazine (S-HyNic), and (4) conjugation of HyNic-IgG to (BSA-4FB)-fluorophore.

The following is a representative example:

Step 1) 4FB modification of BSA: BSA was exchanged in Modification Buffer by dialysis. To a solution of BSA (7 mg; 0.933 mL of a 7.5 mg/mL solution) was added sulfo-S-4FB (5.14 μL of a 18 mg/mL solution in anhydrous DMSO; 5 mol equivalents). The reaction mixture was incubated at room temperature for 2 h and desalted and buffered exchanged using 2 2.0 mL 7K MWCO Zeba columns pre-equilibrated with Modification Buffer.

Step 2) Fluorophore modification of 4FB-BSA: To a solution of 4FB-BSA in Modification Buffer (0.33 mg; 83 μL of a 4 mg/mL solution) was added Dy488-NHS ester (21 μL of a 2.5 mg/mL solution in anhydrous DMSO; 10 mol equivalents). The reaction mixture was incubated at room temperature for 2 hours and desalted twice using 0.5 mL 40 K MWCO Zeba columns pre-equilibrated with Conjugation Buffer.

Step 3) HyNic modification of antibody: To a solution of anti-mouse antibody in Modification Buffer (500 μg; 125.0 μL of a 4.0 mg/mL solution) was added a solution of S-HyNic (3.63 μL of a 4.0 mg/mL solution in DMSO; 15 mol equiv). The reaction mixture was incubated at room temperature for 2 h and desalted using a 0.5 mL 7K MWCO Zeba column pre-equilibrated with Conjugation Buffer. The HyNic-modified antibody was used immediately in the following conjugation reaction.

Step 4) HyNic-antibody/(BSA-4FB)-fluorophore conjugation: To a solution of HyNic-anti-mouse antibody (300 μg; 75 μL of a 4.0 mg/mL solution in Conjugation Buffer) was added (BSA-Dy488)-4FB (0.74 mg; 88.0 μL of a 8.45 mg/mL solution in Conjugation Buffer; 4 mol equivalents) followed by the addition of aniline buffer (16.3 μL). The reaction mixture was incubated at room temperature for 1 h then 4° C. for 16 h. The reaction mixture was buffer exchanged into PBS using a 2.0 mL 7K MWCO Zeba column. Polyacrylamide gel electrophoresis (4-12% Bis-Tris (Novex); MOPS buffer) indicated >90% incorporation of antibody into conjugate. The product was used directly in immunohistochemical staining.

As would be understood by those of ordinary skill in the art, step 1 in the above scheme should be performed with a limiting amount of the amino-reactive 4-formylbenzoate reagent, so that amino groups remain unreacted on the BSA or other coupling protein following this step. The unreacted amino groups are thus free to react in step 2 with the amino-reactive fluorophore. In the exemplary reactions, the BSA-Dy488 product contained about 3-5 dyes per protein, and the BSA-Dy550 and Dy650 products contained about 2-4 dyes per protein.

It should also be understood that the naming convention used for the high-affinity immunopolymers of the instant disclosure may provide descriptive information about the higher-order structure of the immunopolymer. For example, in the just-described immunopolymer, the BSA is labeled with a fluorophore (FL) prior to the association of the coupling protein (e.g., BSA) and the antibody (e.g., IgG) with the high-efficiency conjugation moiety, creating [(BSA-FL)-IgG], for example as shown in FIG. 1. Other exemplary immunopolymers, as described in more detail below, include [(IgG-BSA)-FL], where the fluorophore is attached to the immunoconjugate after the coupling protein (e.g., BSA) and the antibody (e.g., IgG) are associated by the high-efficiency conjugation moiety. Such naming conventions are not absolute, however, and variation in the higher-order structure of the immunoconjugates is considered within the scope of the invention, regardless of the naming of the immunoconjugates, as limited only by the claims.

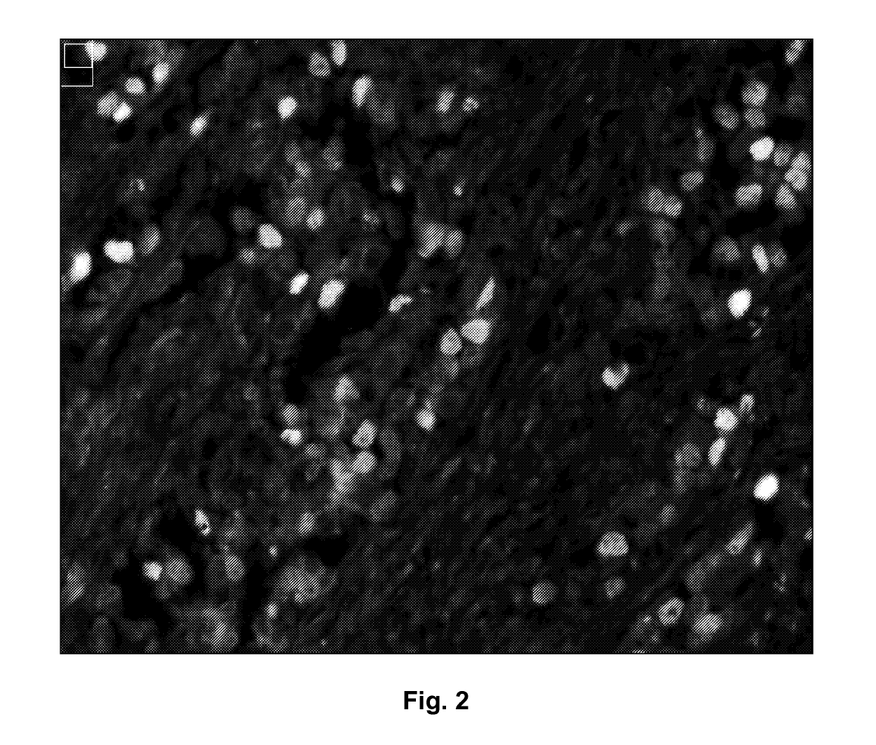

FIG. 2 shows the result of staining using a [(BSA-Dy488)-anti-rabbit] immunopolymer synthesized using HyNic chemistry as described above. Progesterone receptor (PR) was visualized on PR+ breast cancer tissue by initially incubating the tissue with a rabbit anti-PR antibody followed by incubation with [(BSA-Dy488)-anti-rabbit] immunopolymer. Specific staining of PR+ cell nuclei is visible.

### Example 4: Synthesis of [(IgG-BSA)-Fluorophore] Immunopolymer Using MOA Conjugating Reagent

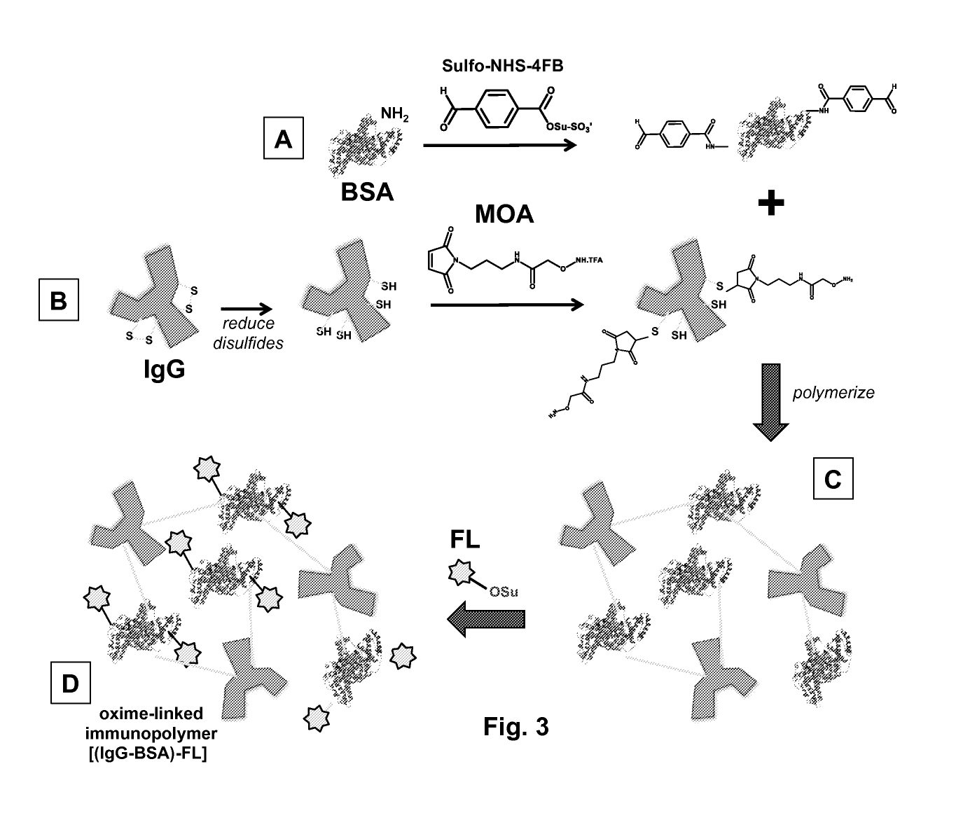

An exemplary synthesis of [(IgG-BSA)-fluorophore] immunopolymers using the MOA conjugating reagent is schematically represented in FIG. 3. The steps include (A) modification of BSA with an 4FB (as in step 1 of Example 3), resulting in an aldehyde modification for the polymerization reaction, (B) controlled reduction of disulfide bonds in the hinge region of the IgG antibody and incorporation of MOA conjugating reagent, resulting in an oxyamino modification for the polymerization reaction, (C) oxime-linked polymerization of MOA-modified IgG to 4FB-BSA, and (D) fluorophore labeling of the (IgG-BSA) polymer to create [(IgG-BSA)-FL] immunopolymer.

The following is a representative example:

Step 1) 4FB modification of BSA: To a solution of BSA (5.0 mg; 0.300 mL of 10 mg/mL solution in Modification Buffer) was added sulfo-succinimidyl 4-formylbenzoate (6.4 μL of a 10 mg/mL solution in DMSO; 4 mol equivalents). The reaction mixture was incubated at room temperature for 2 h and desalted using a 2.0 mL 7 kD MWCO Zeba column pre-equilibrated with Conjugation Buffer. BSA concentration (8.7 mg/mL; BCA Assay); 4FB DOL 1.6 (4-hydrazinopyridine colorimetric assay).

Steps 2 and 3) Reduction of antibody and modification with maleimido-oxyamino (MOA) conjugating reagent: To a solution of goat anti-mouse antibody (931 mg; 186 μL of a 5.0 mg/mL solution in Modification Buffer, pH 7.65, 5 mM EDTA) was added TCEP (9.3 μL of a 21 mM solution in water). The reaction was incubated at room temperature for 1 h followed by the addition of maleimido-oxyamino TFA conjugating reagent (8.54 μL of a 5 mg/mL solution in DMF; 20 mol equivalents). The reaction mixture was further incubated for 1 h and desalted and buffer exchanged using a 0.5 mL 7K MWCO Zeba column pre-equilibrated with Conjugation Buffer/5 mM EDTA.

Step 4) Conjugation of oxyamino-modified antibody to 4FB-BSA: To a solution of oxyamine-modified anti-mouse antibody from Step 1 (877 mg; 205 μL of a 4.28 mg/mL solution in Conjugation Buffer/5 mM EDTA) was added a solution of 4FB-BSA (1.54 mg; 193 μL of a 8.0 mg/mL solution in Conjugation Buffer) and aniline buffer (40 μL). The reaction mixture was incubated overnight at 4° C. and the co-polymer was isolated by size exclusion chromatography using a SuperDex200 column using modification buffer as eluant. The product was analyzed by polyacrylamide gel electrophoresis (4-12% Bis-Tris (Novex).

Step 5) Modification of IgG-BSA co-polymer with fluorophores: To a solution of anti-mouse-BSA co-polymer prepared from Step 4 (nominal MW 330,000 kDa; 0.15 mg; 43 μL of a 3.47 mg/mL solution in Modification Buffer) was added Dy550-OSu (7.1 82 L of a 5.0 mg/mL solution in DMSO; 75 mol equivalents). The reaction mixture was incubated at room temperature for 2 h, diluted with PBS (100 μL) and desalted two times using 40 kD MWCO 0.5 mL Zeba columns pre-equilibrated with PBS. UV analysis determined that 17.2 mol fluorophores were incorporated for the polymer of average MW of 330,000 kDa.

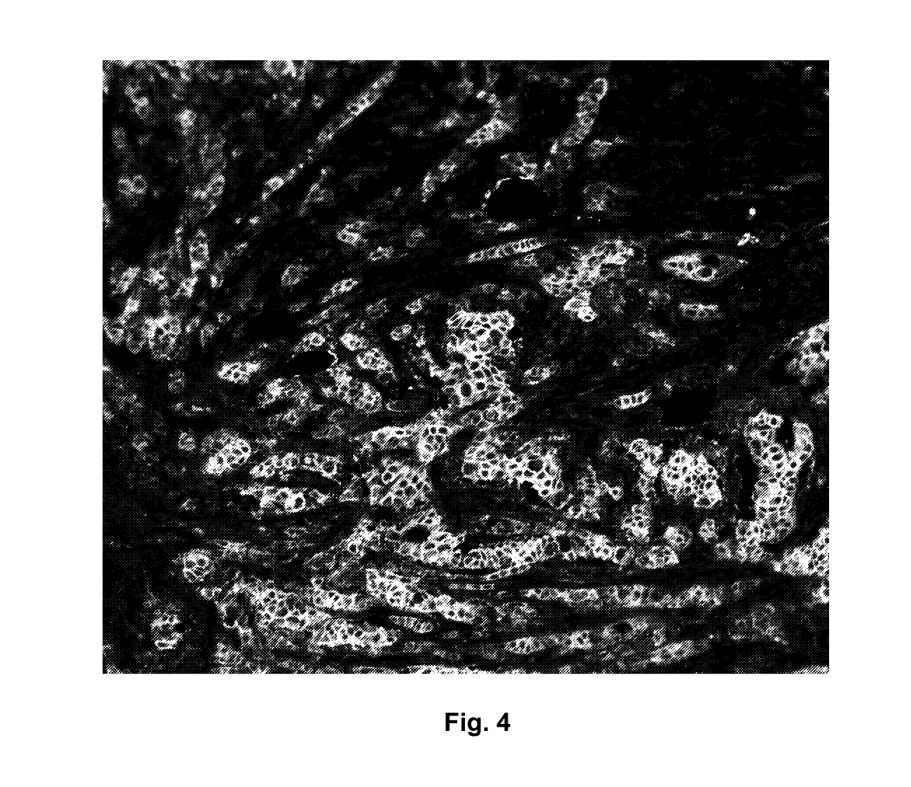

FIG. 4 presents results of staining of Her2+ breast cancer tissue with anti-Her2 primary antibody from rabbit (Rb) followed by staining with [(anti-Rb-BSA)-Dy550] immunopolymer that was prepared by incorporation of multiple oxyamino groups using the MOA conjugating reagent on a reduced goat anti-rabbit IgG as described above. The results demonstrate strong staining of Her2 on the triple-positive breast cancer tissue by the [(anti-Rb-BSA)-Dy550] immunopolymer.

### Example 5: Synthesis of [(IgG-BSA)-Fluorophore] Immunopolymer Using AOA Conjugating Reagent

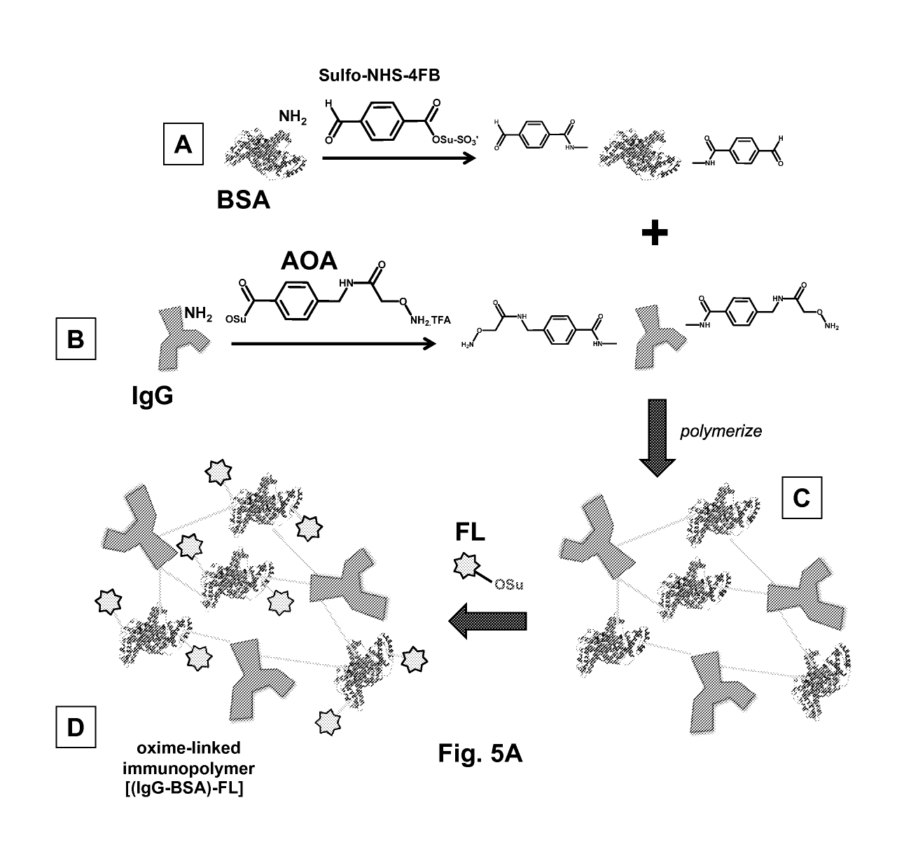

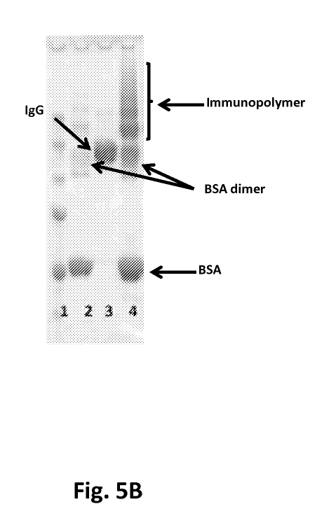

An alternative synthesis of [(IgG-BSA)-fluorophore] immunopolymers using the amino-reactive NHS-AOA conjugating reagent and conjugation to BSA-4FB is illustrated schematically in FIG. 5A. In this example, anti-digoxigenin (DIG) antibody was conjugated with BSA as the coupling protein. The steps include (A) modification of BSA with an 4FB (as in step 1 of Examples 3 and 4), (B) modification of amino groups on IgG using the AOA conjugating reagent, resulting in an oxyamino-modified IgG for polymerization reaction, (C) oxime-linked polymerization of AOA-modified IgG to 4FB-BSA, and (D) fluorophore labeling of the (IgG-BSA) polymer to create [(IgG-BSA)-FL] immunopolymer.

The reaction was carried out as follows:

Step 1) Modification of antibody with amino-reactive oxyamine conjugating reagent: To a solution of anti-digoxigenin antibody (1.200 mg; 240 μL of a 5.0 mg/mL solution in Modification Buffer; Jackson ImmunoResearch, West Grove, Pa.) was added the amino-reactive oxyamine conjugating reagent (AOA) (10.2 μL of a 5.0 mg/mL solution in DMSO; 15 mol equiv). The reaction mixture was incubated for 2 h, desalted and buffer exchanged using a 0.5 mL 7K MWCO Zeba column pre-equilibrated with Conjugation Buffer.

Step 2) Conjugation of oxyamino-anti-DIG antibody to 4FB-BSA: To a solution of oxyamino-modified antibody from Step 1 (1.1 mg; 250 μL of 4.4 mg/mL solution) was added 4FB-BSA (1.88 mg; 243.7 μL of a 8.0 mg/mL solution in Conjugation Buffer; 4 equivalents) (see previous examples) and aniline buffer (48.5 μL). The reaction mixture was incubated overnight at 4° C. The polymer was purified on a SuperDex 200 column eluting with Modification Buffer. The fractions containing the polymer were collected, combined and concentrated using a 7K MWCO diafiltration apparatus. Table 1 lists the antibodies polymerized using this procedure and % yields of recovered antibody.

Step 3) Modification of IgG-BSA co-polymer with fluorophores: To a solution of anti-DIG-BSA co-polymer prepared in Step 2 (nominal MW 330,000 kD; 0.50 mg; 11 μL of a 4.74 mg/mL solution in Modification Buffer) was added amine-reactive Dy650 (1.62 μL of a 5.0 mg/mL solution in DMSO; 50 mol equivalents). The reaction mixture was incubated at room temperature for 2 h, diluted with PBS (50 μL) and desalted two times using 40 kD MWCO 0.5 mL Zeba columns pre-equilibrated with PBS. The fluorophore-labeled immunopolymer was analyzed by UV spectroscopy. Table 1 provides the DOL for the three immunopolymers prepared. An SDS-PAGE of a representative synthesis performed according to this procedure is shown in FIG. 5B.

TABLE 1
Antibodies and fluorophores used to prepare [(IgG-
BSA)-FL] immunopolymers. The % yields are based
on the amount of starting antibody.

| Antibody | Manufacturer | Fluorophore | % Yield | DOL |
|---|---|---|---|---|
| anti-mouse | ImmunoReagents | Dy488 | 98% | 25.7 |
| anti-mouse | ImmunoReagents | Dy550 | 98% | 20.3 |
| anti-mouse | ImmunoReagents | Dy650 | 98% | 21.1 |
| anti-rabbit | ImmunoReagents | Dy488 | 98% | 24.9 |
| anti-rabbit | ImmunoReagents | Dy550 | 98% | 21.4 |
| anti-rabbit | ImmunoReagents | Dy650 | 98% | 22.2 |
| anti-BIO | Jackson Immuno | Dy488 | 95% | 20.1 |
| anti-DIG | Jackson Immuno | Dy650 | 98% | 24.3 |
| anti-DNP | Life Technologies | Dv550 | 86% | 15.0 |

DOL = Degree of fluorophore labeling of immunopolymer, fluorophores-per-polymer (average).

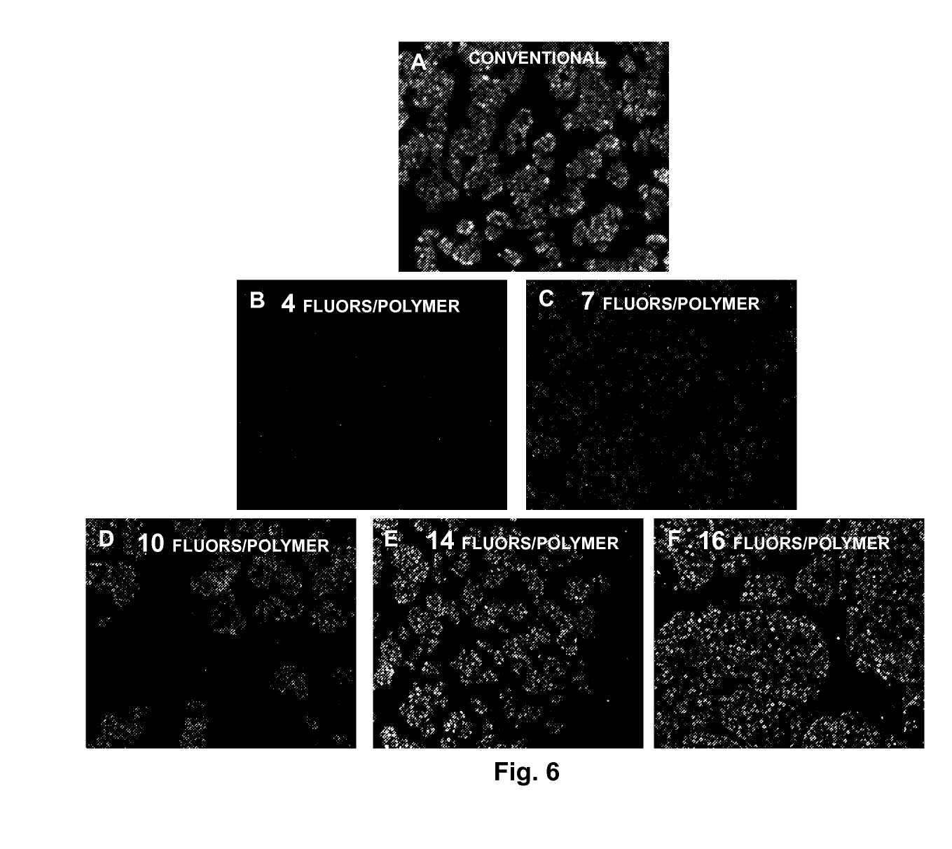

FIG. 6 presents staining data showing the effect of increasing fluorophore incorporation on immunopolymer staining. Comparison of (A) conventional staining of estrogen receptor (ER) on ER+ breast cancer tissue using anti-ER primary antibody from rabbit (Rb) followed by staining with conventional anti-Rb-Dy650 antibody; or (B-F) followed by staining with [(anti-Rb-BSA)-Dy650] having increasing levels of average fluorophore incorporation: (B) 4.2 (C) 6.6 (D) 10.1 (E) 14.1 (F) 16.4 fluorophores per immunopolymer. The immunopolymers were created using the AOA conjugating reagent.

The results clearly demonstrate increasing signal with increasing degree of labeling (DOL) of fluorophores-per-polymer. In addition, at the highest DOL, the generated signal is greater than a commercially available, unconjugated anti-Rb-Dy650 reagent according to evaluation using quantitative imaging software (data not shown).

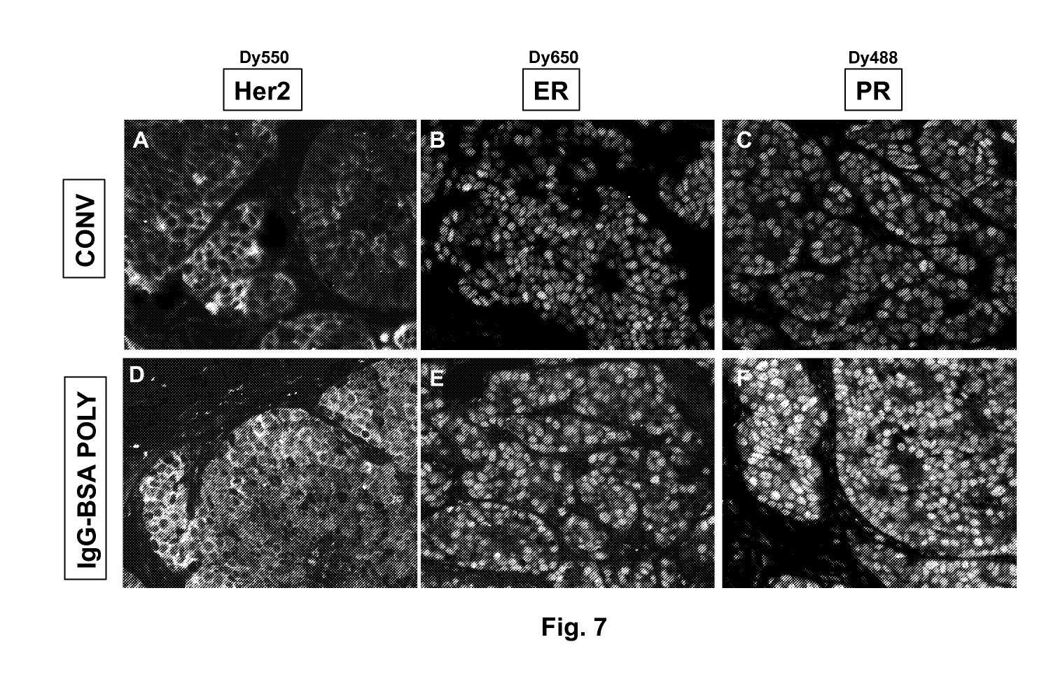

FIG. 7. shows IHC results comparing the sensitivity of conventional fluorescent secondary antibody to novel fluorescent immunopolymers created using the AOA conjugating reagent. Triple-positive (Her2+ER+PR+) breast cancer tissue was stained with either anti-Her2 (A, D) anti-ER (B, E) or anti-PR (C, F), each from rabbit (Rb). The top row (A-C) shows conventional secondary antibody staining using anti-Rb in three fluorescent colors (Dy550, Dy650, or Dy488). The lower row (D-F) shows secondary staining using novel immunopolymers [(anti-Rb-BSA)-FL)] in three fluorescent colors as shown. Results indicate staining with the immunopolymers to be comparable (Her2, ER) or superior to (PR) staining with existing conventional reagents using identical staining conditions and tissue samples.

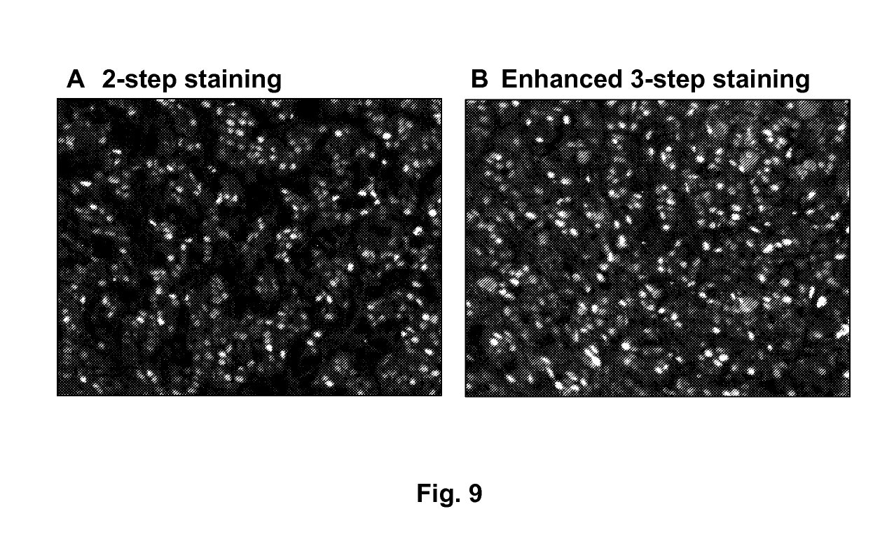

FIG. 9 shows the utility of [(IgG-BSA)-FL] immunopolymers for multiple staining steps, affording enhanced immunofluorescence using three-step immunofluorescent staining. (A) As a control, ER+breast cancer tissue stained in two steps with rabbit anti-ER antibody followed by [(goat anti-rabbit-BSA)-Dy650] immunopolymer. (B) Enhanced staining is accomplished in three steps by staining the anti-ER probed tissue second with [(goat anti-rabbit-BSA)-Dy650] followed by a third staining with [(anti-goat-BSA)-Dy650] immunopolymer.

### Example 6: Synthesis of [Poly(BSA)-Fluorophore]-IgG Conjugates

[(Poly(BSA)-FL)-IgG] immunopolymers were prepared using the following 3-step procedure (1) co-polymerization of BSA, (2) incorporation of fluorophores on the (BSA-BSA) co-polymer and (3) conjugation of IgG to the (BSA-BSA) co-polymer.

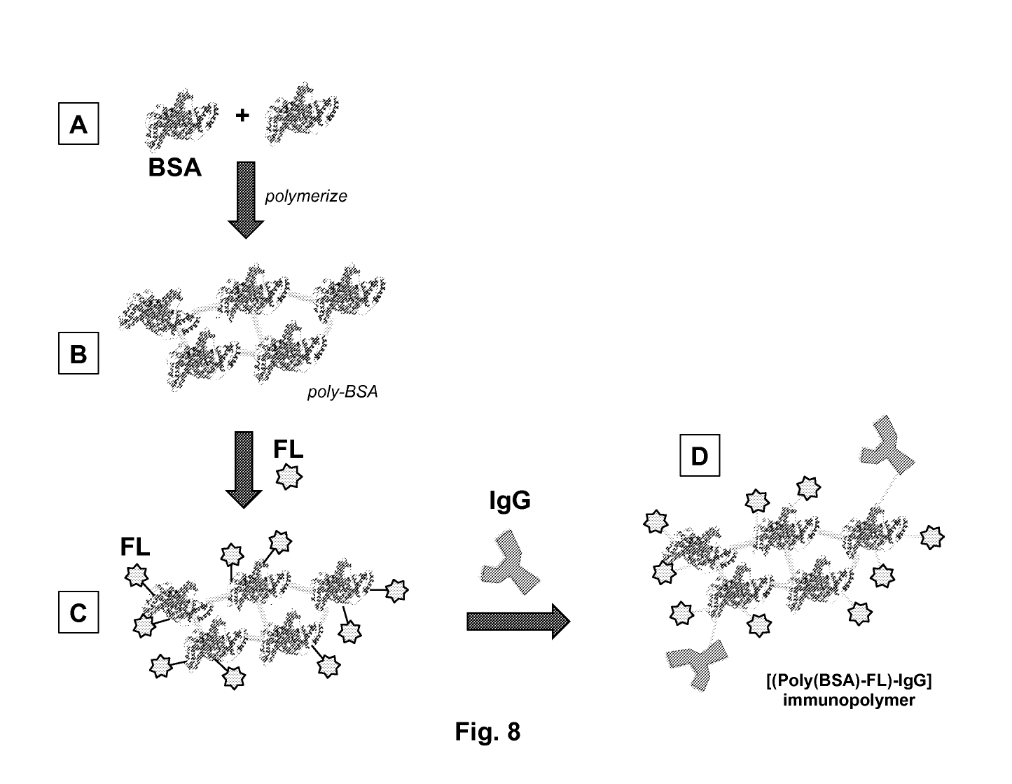

The following protocol, schematically presented in FIG. 8, is a representative example:

Step 1A) Activation of BSA with 4FB: To a solution of BSA (10.0 mg; 0.600 mL of 10 mg/mL solution in Modification Buffer) was added sulfo-succinimidyl 4-formylbenzoate (12.7 μL of a 10 mg/mL solution in DMSO; 4 mol equivalents). The reaction mixture was incubated at room temperature for 2 h and desalted using a 2.0 mL 7 kD MWCO Zeba column pre-equilibrated with Conjugation Buffer. BSA concentration (8.7 mg/mL; BCA Assay); 4FB DOL 1.6 (4-hydrazinopyridine colorimetric assay)

Step 1B) Activation of BSA with S-HyNic: To a solution of BSA (10.0 mg; 1.00 mL of 10 mg/mL solution in Modification Buffer) was added succinimidyl 6-hydrazinonicotinate acetone hydrazone (35.1 μL of a 5.0 mg/mL solution in DMSO; 4 mol equivalents). The reaction mixture was incubated at room temperature for 2 h and desalted using a 2.0 mL 7 kD MWCO Zeba column pre-equilibrated with Conjugation Buffer. BSA concentration (9.1 mg/mL; BCA Assay) HyNic DOL 3.5; (sulfo-benzaldehyde colorimetric MSR assay).

Step 2) Conjugation of BSA-4FB to BSA-HyNic: To a BSA-4FB (1.0 mg; 115 μL of a 8.7 mg/mL solution in Conjugation Buffer) was added BSA-HyNic (3 mg; 329.7 μL of a 9.10 mg/mL solution in Conjugation Buffer; 3 mol equivalents) and aniline buffer (44.5 μL). The reaction mixture was incubated overnight at room temperature and purified by size exclusion HPLC on a SuperDex 200 column eluting with Modification Buffer at 0.5 mg/mL.

Step 3) Fluorophore modification of the Poly(BSA) co-polymer: To a solution of (BSA-BSA) co-polymer (nominal molecular weight 200,000 kD; 140 μg of a 10 mg/mL solution in Modification Buffer) was added Dy650-OSu (1.5 μL of a 5.0 mg/mL solution in DMSO; 10 mol equiv). The reaction mixture was incubated at room temperature for 2 h and desalted twice using 0.5 mL 40 kD MWCO Zeba column pre-equilibrated with Conjugation Buffer. Dy500 DOL 4.52 (UV determination)

Step 4A) HyNic modification of anti-rabbit IgG: To a solution of anti-ER IgG (0.40 mg; 200 μL of a 2.0 mg/mL solution in Conjugation Buffer) was added a solution of S-HyNic (3.87 μL; 200 μL of a 2.0 mg/mL solution in DMF; 10 mol equiv). The reaction mixture was incubated at room temperature for 2 h, and desalted two times using 40 kD MWCO 0.5 mL Zeba columns pre-equilibrated with Conjugation Buffer.

Step 4B) Activation of Poly(BSA) co-polymer with 4FB: To a solution of (BSA-BSA) co-polymer prepared in Step 3 (1.0 mg; 0.100 mL of 10 mg/mL solution in Modification Buffer) was added sulfo-succinimidyl 4-formylbenzoate (0.35 μL of a 8 mg/mL solution in DMSO; 4 mol equivalents). The reaction mixture was incubated at room temperature for 2 h and desalted using a 2.0 mL 7 kD MWCO Zeba column pre-equilibrated with Conjugation Buffer.

Step 5) Conjugation of HyNic anti-ER to 4FB-Poly(BSA): To a solution of Poly(BSA)-Dy650-4FB (35 μg; 7 μL of a 5 mg/mL solution in Conjugation Buffer; 0.175 nmol; nominal molecular weight 200 kD) was added a solution of anti-ER-HyNic (26 μg; 14.6 μL of a 1.8 mg/mL solution in Conjugation Buffer; 0.175 nmol) and aniline buffer (2.2 μL). The reaction was incubated overnight at 4° C. The reaction mixture was desalted using a 0.5 mL Zeba column pre-equilibrated with PBS. The isolated product was used without further purification.

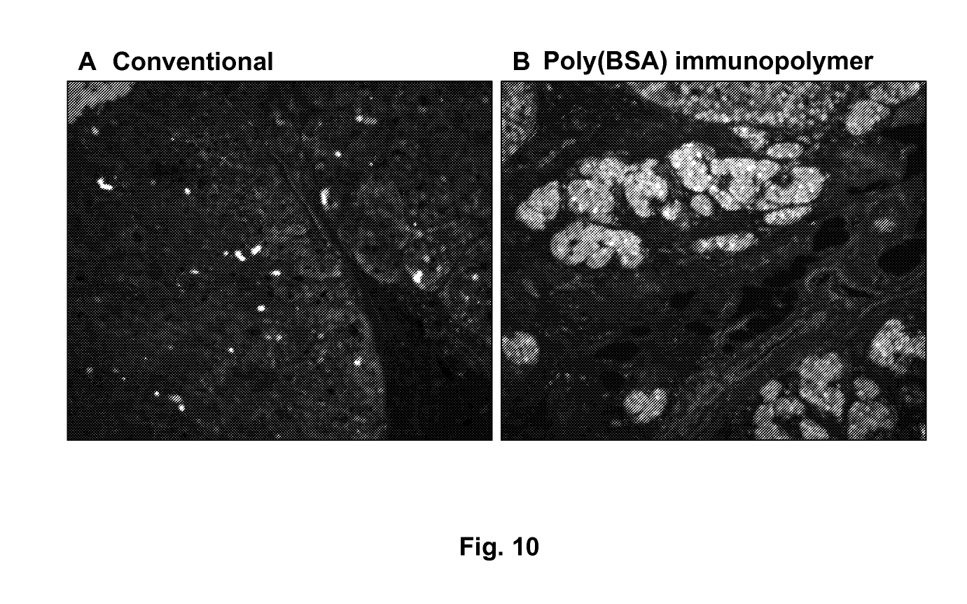

FIG. 10 shows direct antigen staining using Poly(BSA) immunopolymer. ER+ breast cancer tissue was either (A) stained conventionally with an anti-ER antibody (from Rb) followed by conventional anti-Rb-Dy650; or (B) directly stained using [(PolyBSA-Dy650)-anti-ER] immunopolymer prepared as shown in FIG. 8. The results clearly demonstrate that the conjugate stains the tissue with greater sensitivity than the standard commercially available anti-rabbit-Dy650 antibody.

### Example 7: Triplex Staining Using Hapten-Labeled Antibody with [(Anti-Hapten-BSA)-Fluorophore] Immunopolymer Pairs

In this example three target antigens were detected on a single tissue using hapten-labeled antibody with [(anti-hapten-BSA)-fluorophore] immunopolymer pairs. Initially three different haptens—dinitrophenyl (DNP), digoxigenin (DIG), and biotin (BIO)—were conjugated to three primary antibodies (anti-Her2, anti-ER, and anti-PR, respectively), as described below. Three [(anti-hapten-BSA)-fluorophore] immunopolymers were prepared, purified, and fluorescently labeled as described in Example 5. Specifically, [(anti-BIO-BSA)-Dy488], [(anti-DNP-BSA)-Dy550] and [(anti-DIG-BSA)-Dy650] immunopolymers were prepared.

Hapten labeling of rabbit monoclonal antibody: The following protocol was used to conjugate DNP to anti-Her2 antibody. Similar protocols were used to conjugate the other haptens to their respective antibodies. To a solution of anti-Her2 (78 μL; 60 μg@0.8 mg/mL; 0.4 nmol) in Modification Buffer was added a solution of DNP-Peg4-OSu (0.68 μL of a 5.0 mg/mL solution in DMSO; 6.4 nmol; 16 mol equiv; QuantaBiodesign, Columbus, Ohio). The reaction was incubated at room temperature for 2 h and desalted into Modification Buffer using a 0.5 mL Zeba column pre-equilibrated with Modification Buffer. The antibody concentration and number of haptens incorporated, 3.47 hapten/antibody, were quantified by measuring the A₂₈₀ and A₃₆₀ (molar extinction coefficient 17,000). Biotin was incorporated on anti-PR using ChromaLink Biotin (Solulink, San Diego, Calif.) and digoxigenin was incorporated on anti-ER using ChromaLink Digoxgenin (Solulink).

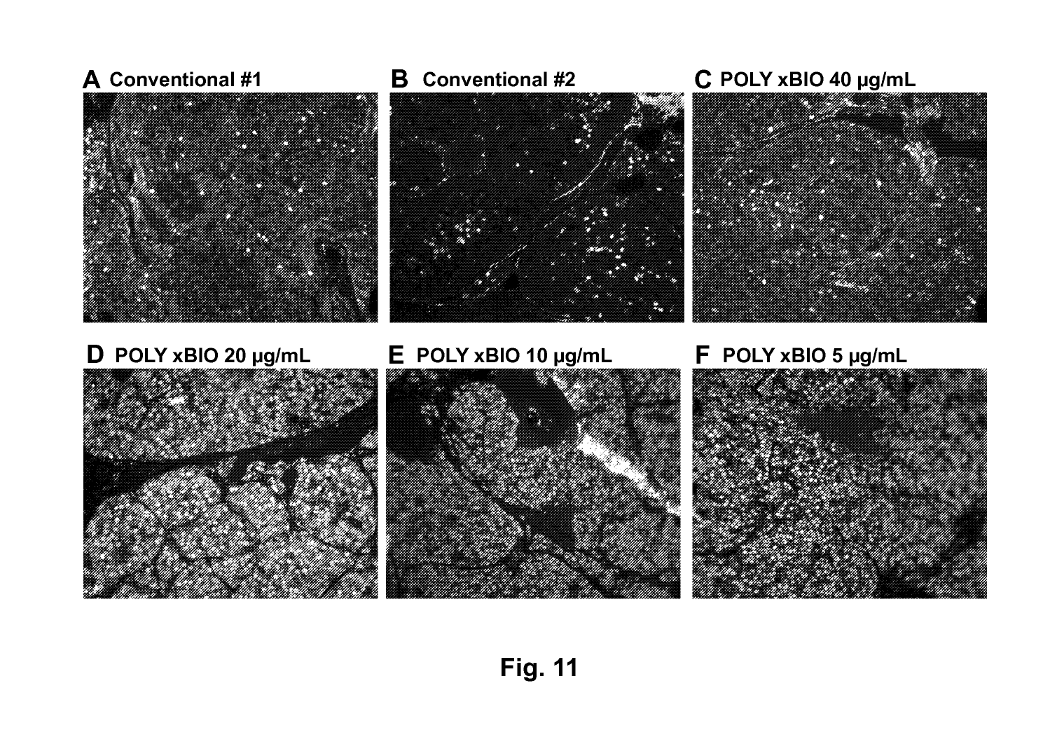

In FIG. 11, effect of immunopolymer staining concentration on [(anti-hapten-BSA)-FL] is shown. Using biotin (BIO) as the hapten, PR+ breast cancer tissue was stained using the following combinations: (A) conventional non-hapten approach, unlabeled anti-PR with conventional anti-Rb-Dy488; (B) conventional biotin/anti-biotin hapten approach, using anti-PR-biotin with commercial anti-biotin-Dy488; (C-F) hapten approach using novel immunopolymer [(anti-biotin-BSA)-Dy488]. A range of immunopolymer concentrations were tested: (C) 40 μg/mL (D) 20 μg/mL (E) 10 μg/mL and (F) 5 μg/mL. Lower intensity signal generated at higher immunopolymer concentrations (C) is ascribed to quenching of the fluorescent signal at the higher concentrations. For many concentrations tested (20, 10, 5 μg/mL) the immunopolymer outperformed either conventional staining approach.

Triplex hapten-mediated immunofluorescence staining—a single formalin-fixed paraffin-embedded (FFPE), triple-positive (He2+ER+PR+) breast cancer tissue sample was stained as detailed in the immunofluorescence protocol above. Each primary antibody had been previously titrated to determine its optimal staining concentration. Specifically, to the tissue section was added a solution of anti-Her2-DNP (2.5 μg/mL), anti-ER-DIG (10 μg/mL), and anti-PR-BIO (10 μg/mL), in 150 μL of antibody diluent and incubated overnight at 4° C. The slide was washed, and a solution of [(anti-DNP-BSA)-Dy550], [(anti-DIG-BSA)-Dy650], and [(anti-BIO-BSA)-Dy488] immunopolymers (5 μg/mL each) was prepared, added to the tissue sections, and incubated at room temperature for 1 h. Slides were washed again, and tissue was mounted under a cover glass for imaging.

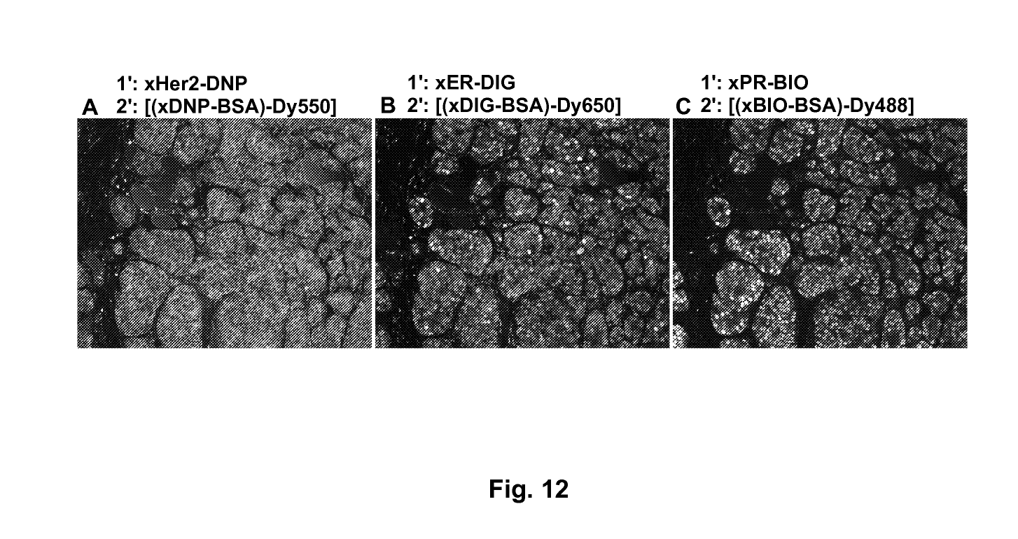

FIG. 12 presents the results of triplex staining. Results are shown presenting (A) Her2 staining in Dy550 fluorescent channel; (B) ER staining in Dy650 channel; and (C) PR staining in Dy488 channel. Antigen-specific staining is clearly visible in each fluorescent channel. Because it is not readily feasible to triple-stain tissue using existing reagents, a conventional comparison is not shown.

### Example 8: Synthesis of [(Poly(IgG)-Fluorophore] Immunopolymers

[(Poly(IgG)-fluorophore] conjugates were prepared by incorporation of amino-reactive NHS-AOA conjugating reagent onto a primary antibody and conjugation of the modified primary antibody to non-specific 4FB-bovine IgG.

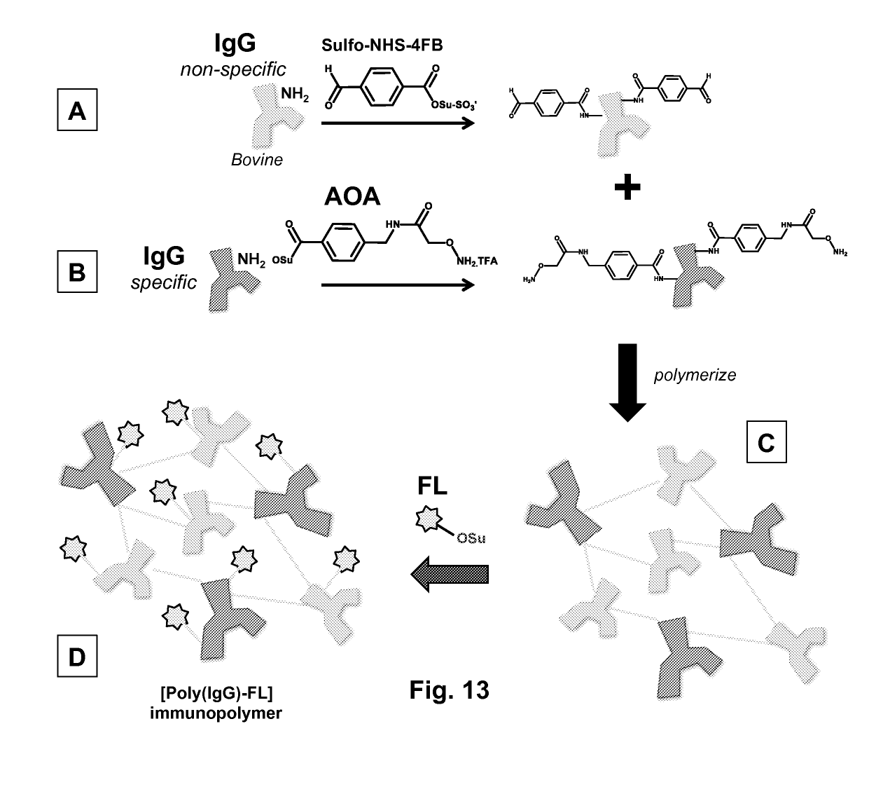

The method, as schematically illustrated in FIG. 13, is exemplified by the following procedure, wherein target-specific goat anti-rabbit IgG is co-polymerized with non-specific bovine IgG (Bov(IgG)):

Step 1) Modification of Bov(IgG) with sulfo-4FB: To a solution of non-specific Bov(IgG) (3.4 mg; 360 μL of a 9.4 mg/mL solution in Modification Buffer) was added sulfo-S-4FB (9.84 μL of a 10.0 mg/mL solution in DMSO; 12.5 mol equiv). The reaction mixture was incubated for 2 h, desalted and buffer exchanged using a 2.0 mL 40K MWCO Zeba column pre-equilibrated with Conjugation Buffer.

Step 2) Modification of IgG with amino-reactive oxyamine conjugating reagent: To a solution of anti-rabbit IgG (anti-Rb(IgG); 0.65 mg; 650 μL of a 10.0 mg/mL solution in Modification Buffer) was added amino-reactive conjugating reagent (55.1 μL of a 5.0 mg/mL solution in DMSO; 15 mol equiv). The reaction mixture was incubated for 2 h, desalted and buffer exchanged using a 2.0 mL 40K MWCO Zeba column pre-equilibrated with Conjugation Buffer.

Step 3) Polymerization of oxyamino-anti-Rb(IgG) to 4FB-Bov(IgG): To a solution of oxyamino-modified anti-Rb(IgG) from Step 2 (0.22 mg; 29 μL of 7.7 mg/mL solution) was added 4FB-BSA (0.44 mg; 55 μL of a 8.0 mg/mL solution in Conjugation Buffer; 2 equivalents) and aniline buffer (8.4 μL). The reaction mixture was incubated overnight at 4° C. There was significant precipitation following incubation. The reaction mixture was centrifuged and the supernatant containing the immunopolymer. The immunopolymer was purified on a SuperDex 200 column eluting with Modification Buffer. The fractions containing the immunopolymer were collected, combined and concentrated using a 7K MWCO diafiltration apparatus. The (anti-Rb(IgG)-Bov(IgG)) immunopolymer product yield was 56 mg.

Step 4) Modification of [anti-Rb(IgG)-Bov(IgG)] immunopolymer with fluorophores: To a solution of [anti-Rb(IgG)-Bov(IgG)] immunopolymer prepared in Step 3 (nominal MW 330,000 kD; 0.56 mg; 37 μL of a 1.52 mg/mL solution in Modification Buffer; 0.19 nmol) was added Dy650-NHS (2.91 μL of a 5.0 mg/mL solution in DMSO; 75 mol equivalents; 14.0 nmol). The reaction mixture was incubated at room temperature for 2 h, and desalted two times using 40 kD MWCO 0.5 mL Zeba columns pre-equilibrated with PBS. The fluorophore-labeled polymer, [Poly(IgG)-FL], was analyzed by UV spectroscopy, indicating the incorporation of 9.5 Dy650/300 kDa.

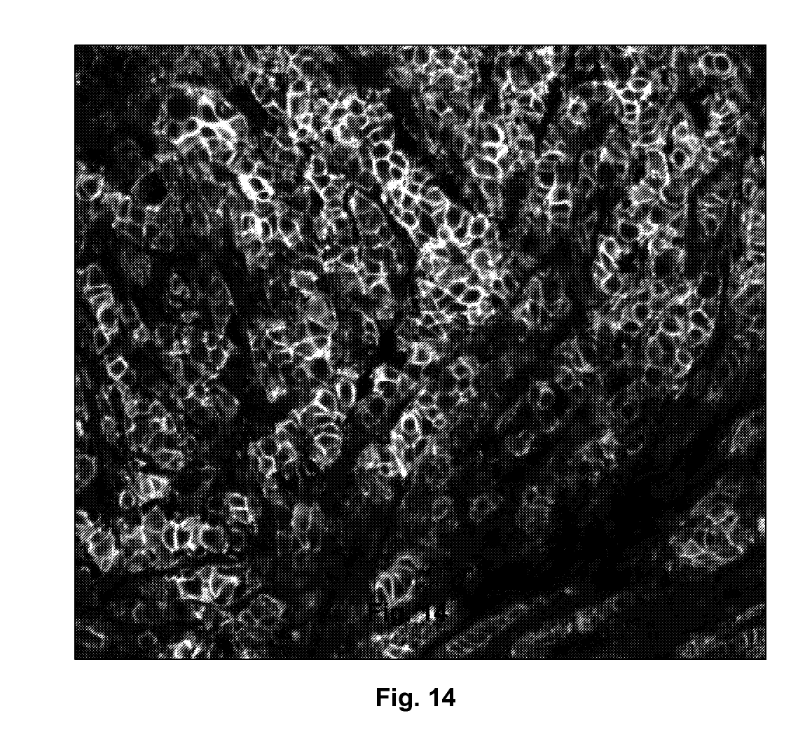

FIG. 14 demonstrates [(Poly(IgG)-FL] immunopolymer staining of Her2 on Her2+ breast cancer tissue with anti-Her2 (Rb) followed by [(anti-Rb(IgG)-Bov(IgG))-Dy550] immunopolymer prepared as described in this example. Highly specific staining of cell membrane target Her2 is visible.

### Example 9: Synthesis of Enzyme-Antibody Immunopolymers and their Use in Immunostaining and Immunoblotting

Conjugation of primary and secondary antibodies to Horseradish Peroxidase (HRP; BBI Solutions, Madison, Wis.) and Soybean Peroxidase (SBP; Bio-Research Products, North Liberty, Iowa) were performed using the following steps: (1) modification of the peroxidase with 4FB, (2) modification of the antibody with amino-reactive NHS-AOA conjugating reagent, and (3) conjugation of peroxidase-4FB to antibody-AOA in the presence of aniline followed by HPLC purification. This workflow is similar to that shown in FIG. 5A, except that the enzyme is used in place of BSA.

### Synthesis of an [(IgG-Poly(HRP)] Immunopolymer

Step 1) HRP-4FB modification: To a solution of HRP (19.8 mg; 1.6 mL of a 12.4 mg/mL in Modification Buffer) was added sulfo-4FB (236 μL of a 10 mg/mL solution in DMSO; 15 equiv). The reaction mixture was incubated at room temperature for 2 h and desalted into Conjugation Buffer using 2 2.0 mL Zeba columns pre-equilibrated with Conjugation Buffer. Assumed a 90% recovery.

Step 2: Modification of antibody with amino-reactive oxyamine conjugating reagent (NHS-AOA): To a solution of anti-rabbit IgG (6.5 mg; 650 μL of a 10.0 mg/mL solution in Modification Buffer) was added an amino-reactive conjugating reagent (compound 13 from Scheme 2; 55 μL of a 5.0 mg/mL solution in DMSO; 15 mol equiv). The reaction mixture was incubated for 2 h, desalted and buffer exchanged using a 0.5 mL 7K MWCO Zeba column pre-equilibrated with Conjugation Buffer.

Step 3: Conjugation of 4FB-HRP to AOA-IgG: To a solution of anti-rabbit IgG-AOA (5.28 mg; 750 μL of a 7.04 mg/mL solution in Conjugation Buffer) was added HRP-4FB (6.2 mg; 620 μL of a 10.0 mg/mL solution in Conjugation Buffer; 4 mol equivalents) and aniline buffer (137 μL). The reaction mixture was incubated overnight at 4° C. and purified by size exclusion HPLC on a SuperDex 200 column eluting with 10 mM phosphate, 150 mM NaCl, pH 7.0.

### Synthesis of an SBP-Antibody Conjugate

Step 1: SBP-4FB modification: To a solution of SBP (Bio-Research Products, North Liberty, Iowa; 2.60 mg; 173 μL of a 15 mg/mL in Modification Buffer) was added sulfo-4FB (31 μL of a 10 mg/mL solution in DMSO; 15 equiv). The reaction mixture was incubated at room temperature for 2 h and desalted into Conjugation Buffer using a 0.5 mL Zeba columns pre-equilibrated with Conjugation Buffer. Assumed a 90% recovery.

Step 2: Modification of antibody with amino-reactive oxyamine conjugating reagent (NHS-AOA): To a solution of anti-rabbit IgG (Immunoreagents, Inc; 0.82 mg; 116 μL of a 7.02 mg/mL solution in Modification Buffer) was added an amino-reactive conjugating reagent (AOA) (compound 13 from Scheme 2; 6.95 μL of a 5.0 mg/mL solution in DMSO; 15 mol equiv). The reaction mixture was incubated for 2 h, desalted and buffer exchanged using a 0.5 mL 7K MWCO Zeba column pre-equilibrated with Conjugation Buffer. Final concentration as determined by A₂₈₀ was 6.12 mg/mL).

Step 3: Conjugation of 4FB-SBP to AOA-IgG: To a solution anti-rabbit IgG-AOA (0.73 mg; 120 μL of a 6.12 mg/mL solution in Conjugation Buffer) was added SBP-4FB (0.86 mg; 86 μL of a 10.0 mg/mL solution in Conjugation Buffer; 4 mol equivalents) and aniline buffer (137 μL). The reaction mixture was incubated overnight at 4° C. and purified by size exclusion HPLC on a SuperDex 200 column eluting with 10 mM phosphate, 150 mM NaCl, pH 7.0. Antibodies conjugated to HRP and/or SBP are listed in Table 2.

TABLE 2
Conjugation of antibodies to enzymes.

| Antibody | Source | Enzyme |
|---|---|---|
| anti-rabbit | Immunoreagents, Inc. | HRP and SBP |
| anti-mouse | Immunoreagents, Inc. | HRP and SBP |
| anti-Ki67 | Biolegend, Inc. | HRP |

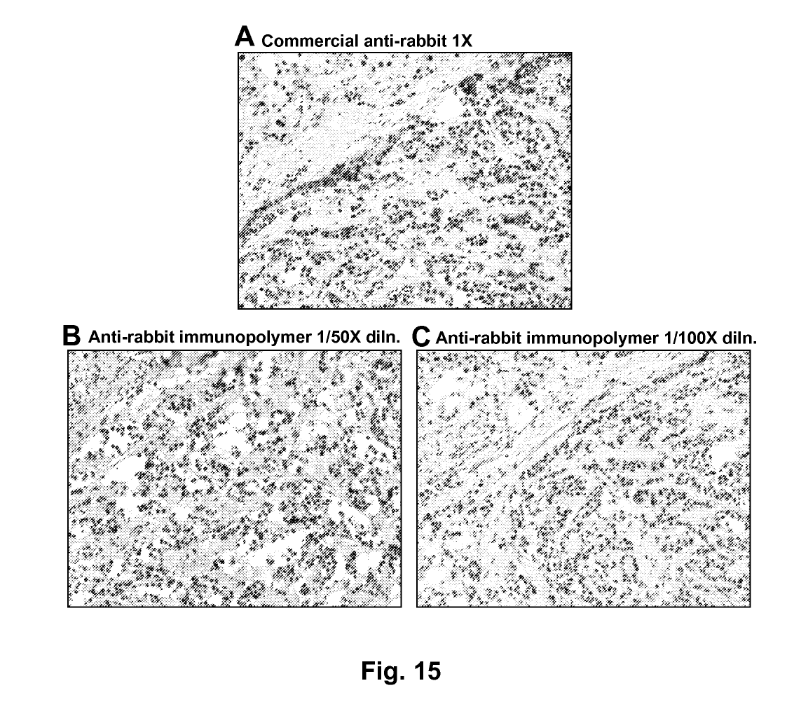

FIG. 15 provides a comparison of a full-strength, undiluted commercial anti-Rb-HRP secondary antibody to diluted [(anti-Rb-Poly(HRP)] immunopolymer. ER+ breast cancer tissue was first stained using anti-ER (Rb) followed by either (A) a commercial anti-Rb polymer at 1× (“PowerVision”, Leica Microsystems, Buffalo Grove, Ill.; ˜0.5 mg/mL) as provided by manufacturer; or a diluted [(anti-Rb-HRP)] immunopolymer: (B) 1/50× dilution, ˜20 μg/mL; (C) 1/100× dilution; ˜10 μg/mL, thus demonstrating similar sensitivity of staining at significantly lower concentration of the HRP-antibody conjugate.

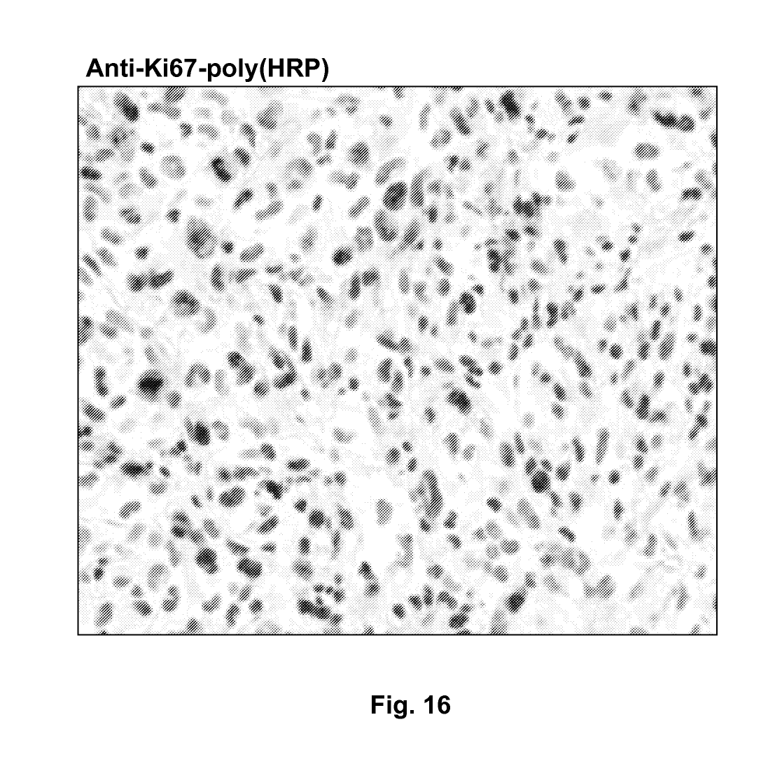

FIG. 16 shows immunostaining using a primary-antibody enzymatic immunopolymer. Ki67+ breast cancer tissue was stained with primary [(anti-Ki67-poly(HRP)] immunopolymer. 3,3′-diaminobenzidine (DAB) chromogenic HRP enzyme substrate was then used to visualize HRP on tissue (dark grey). Nuclei were counterstained with hematoxylin (light grey). As expected, the majority of the nuclei in the image stained Ki67+. Ki67-stained nuclei were not visible in Ki67-negative tissue (data not shown).

### ‘Western’ Immunoblotting Using HRP and SBP Enzyme Immunopolymers

Antibody-HRP enzyme conjugates are commonly used to visualize proteins immobilized in synthetic immunoblotting membranes. By applying an enzyme-reactive chemiluminescent substrate, a signal can be captured on film to qualify the presence of target protein in a given sample. This technique is widely practiced in molecular biology research laboratories.

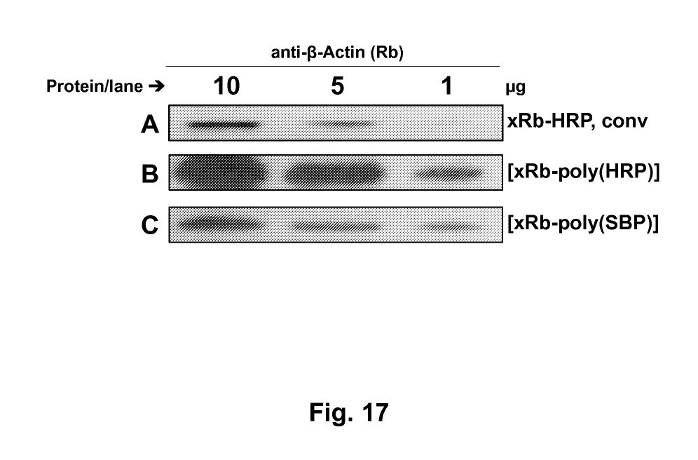

FIG. 17 compares the performance of conventional antibody-HRP compared to antibody-enzyme immunopolymers prepared using the AOA conjugating reagent. To evaluate all antibody-enzyme conjugates using the same conditions, all samples were comprised of standard preparations of murine cellular lysate probed for the ubiquitous cellular protein beta-actin. Cellular lysate was prepared from cultured melanoma cells using standard methods and assayed for total protein content using BCA protein assay.

Protein lysate was loaded into acrylamide gels for separation by molecular weight using conventional electrophoresis methods (SDS-PAGE). Decreasing amounts of protein were loaded per lane (10, 5, 1 μg) to enable evaluation of antibody-enzyme detection sensitivity. The acrylamide gels were transferred to nitrocellulose membranes using standard wet-transfer methods.

After blocking all membranes with BSA to prevent nonspecific antibody binding, polyclonal beta-actin antibody from rabbit (Santa Cruz Biotechnology, Santa Cruz, Calif.) was applied equally to all samples, and the membranes were stringently washed. Anti-rabbit enzyme conjugates were applied at 1/10,000× dilution and the membranes were again stringently washed.

Chemiluminescent peroxidase substrate (ECL “Pico”, ThermoPierce, Madison, Wis.) was briefly applied to each membrane followed by exposure of membranes to film. Exposure time was equal for all samples shown.

FIG. 17 shows protein immunoblotting with enzymatic immunopolymers comparing (A) conventional anti-Rb-HRP (GE Life Sciences, Piscataway, N.J.) to (B) [(anti-Rb-Poly(HRP)] or (C) [(anti-Rb-Poly(SBP)] immunopolymers. A decreasing amount of total protein was loaded per lane (10, 5, 1 μg/lane) to test the limits of detection.

These results demonstrate that both enzyme immunopolymers (Poly(HRP) and Poly(SBP)) exhibited higher sensitivity than the conventional antibody-HRP conjugate. At 10 μg and 5 μg per lane, the signal afforded by the antibody-enzyme polymers is at least several-fold more intense than that provided by the conventional antibody-HRP reagent. At 1 μper lane, a signal is visible for samples visualized with antibody-enzyme polymers, but not for the sample with conventional antibody-HRP.

### Example 10: Tyramide Amplification Using Enzyme-Antibody Immunopolymers

The novel antibody-HRP immunopolymers of the instant disclosure have also been demonstrated to have benefits over conventional signal detection methods using tyramide signal amplification. Tyramide signal amplification (TSA) is a strategy employed to enhance staining of cellular components for improved visualization over conventional fluorescent antibody detection or fluorescent streptavidin-biotin signal amplification. TSA utilizes a reaction between a peroxidase enzyme and a haptenylated tyramide reagent, catalyzed by hydrogen peroxide, to locally deposit hundreds to thousands of haptens in the vicinity of the cellular target. The locally deposited haptens are then visualized by a fluorescent hapten-reactive reagent (for example, streptavidin conjugated to DyLight 650).

In this Example, existing TSA methodology is shown to be enhanced by using antibody-HRP immunopolymers prepared using the AOA conjugating reagent as described herein. The antibody-HRP immunopolymers provide enhanced signal amplification by presenting multiple peroxidase sites for tyramide reaction. Conventional TSA and other conventional protein staining methods have also been used for comparison here.

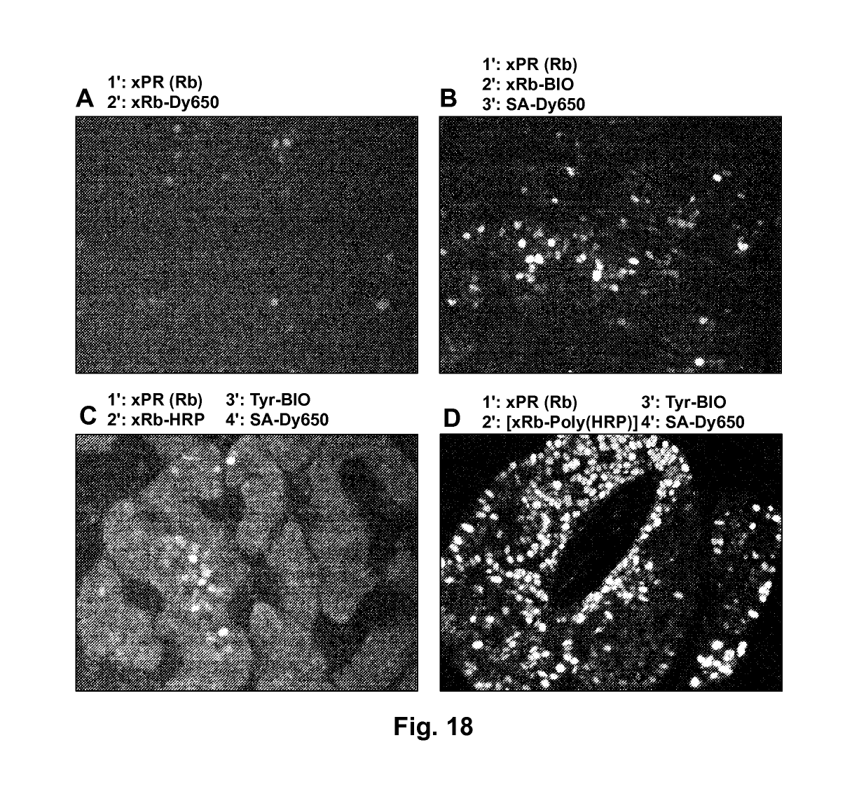

FIG. 18 shows a comparison of enhanced TSA with other methods of signal detection in fixed human tissue. For each staining method shown (A-D), the cellular protein target probed was progesterone receptor (PR). Tissue was handled identically except for staining reagents applied, as noted in FIG. 18. Imaging was conducted using identical settings for each sample. All tissues were PR+ and were first stained with anti-PR (xPR, Rb). (A) Conventional detection using anti-rabbit-Dy650 (xRb-Dy650); (B) conventional signal amplification strategy using biotin/streptavidin, applying first anti-rabbit-biotin (xRb-BIO) followed by streptavidin-Dy650 (SA-Dy650); (C) Conventional TSA strategy using commercial anti-rabbit-HRP (xRb-HRP), followed by commercial biotinylated tyramide (Tyr-BIO, Toronto Research Chemicals, Toronto, Canada) and SA-Dy650; (D) TSA using immunopolymer [anti-Rb-poly(HRP)] followed by Tyr-BIO and SA-Dy650.

The results shown in FIG. 18 demonstrate significant enhancement of TSA staining using the novel antibody-HRP immunopolymer (D). Staining with the novel immunopolymer provides the brightest and most superior signal over background. Conventional antibody staining (A) provided a signal, but the signal was difficult to resolve over background. Staining using the moderate amplification afforded by biotin-streptavidin (B) was more effective, but did not sufficiently label all PR cells in the field of view. In particular, PR-LOW cells were difficult to resolve over background. Conventional TSA staining (C) resulted in extremely high background. As shown in (D), however, enhanced TSA using the novel HRP immunopolymer provided excellent signal, low background, and visible labeling of even PR-LOW cells.

### Example 11: Flow Cytometry with [(Anti-Hapten-BSA)-Fluorophore] Immunopolymers

In this example, novel haptenylated immunopolymers were compared to conventional probes when used to label surface antigens on a particular subset of cells within a complex population of viable cells and subjected to flow cytometry. The example thus demonstrates the utility of the immunopolymers in routine cytometric assays. Specifically, a preparation of human white blood cells was labeled for the common T-cell surface antigen CD3 using three separate approaches, as described in detail below.

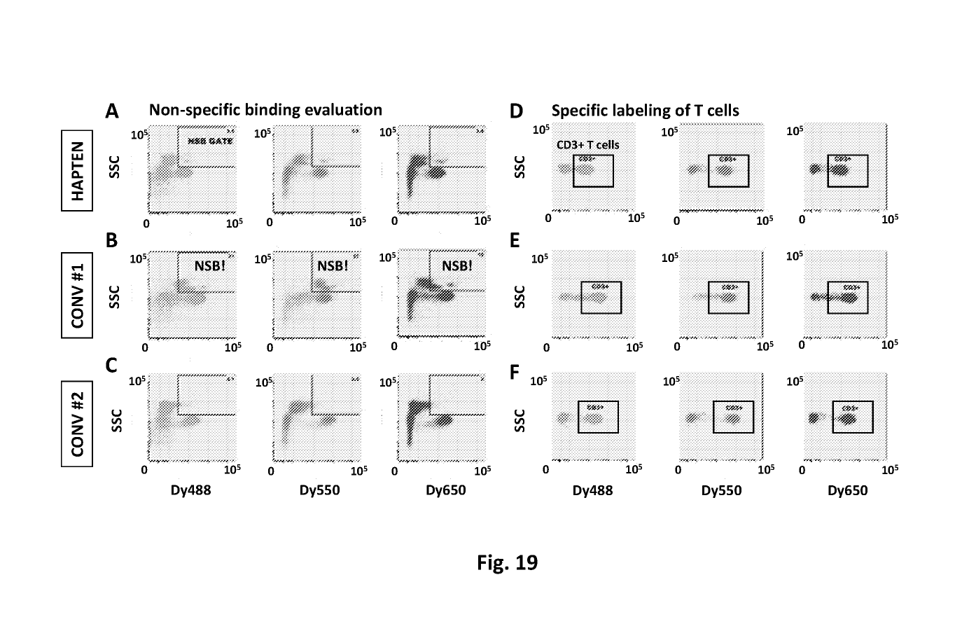

FIG. 19 shows cytometric labeling results comparing novel immunopolymers to conventional probes:

Rows A, D: A first group of cell samples were labeled using a novel haptenylated immunopolymer (anti-CD3: DNP) followed by treatment with a novel fluorescent anti-DNP immunopolymer in each of three fluorescent colors (Dy488; Dy550, and Dy650).
Rows B, E: A second group of cell samples were labeled using unlabeled anti-CD3 (from mouse, Monoclonal Antibody Facility, University of Chicago) followed by a conventional anti-mouse fluorescent secondary probe (ThermoPierce, Asheville, N.C.) in the same three colors noted above.
Rows C, F: A third group of cell samples were labeled using a commercial biotinylated anti-CD3 (Biolegend, San Diego, Calif.) followed by a conventional fluorescent streptavidin probe (ThermoPierce, Asheville, N.C.) in the same three colors noted above. Experimental and analytical procedures were identical for all groups and all samples.

Rows A-C depict cytometric gates indicating non-specific binding (NSB) of monocytes, a population that should exhibit no binding of the probe and should therefore display no fluorescent signal. The conventional secondary anti-mouse probes (Row B) exhibit the highest level of NSB.

Rows (D-F) depict cytometric plots showing gated lymphocytes only, as fluorescent signal vs cellular granularity, with CD3+ cells shown gated. The percentage of CD3+ T-cell lymphocytes is expected to be approximately 70% in each cellular sample. Staining near 70% indicates appropriate reagent performance. Staining<70% indicates under-performance of reagents, and staining>70% indicates nonspecific binding of other cells in the sample (i.e. B-cells, NK cells, etc.), an undesirable outcome.

Results of this experiment indicate that the novel haptenylated immunopolymer plus anti-hapten fluorescent immunopolymers in combination effectively, and specifically, labeled T-cells in a complex cellular sample, and exhibited lower non-specific binding than some commercial reagents (i.e. anti-mouse probes). These results demonstrate the utility and high performance of the haptenylated immunopolymers of the instant disclosure in cytometric applications.

### Example 12: Quantitative Immunohistochemical Staining using Microsphere Calibrants

Microspheres with surfaces haptenlyated at different surface densities are disclosed herein as calibrants for use in a variety of molecular imaging applications. This example describes the preparation and evaluation of exemplary haptenylated micro spheres.

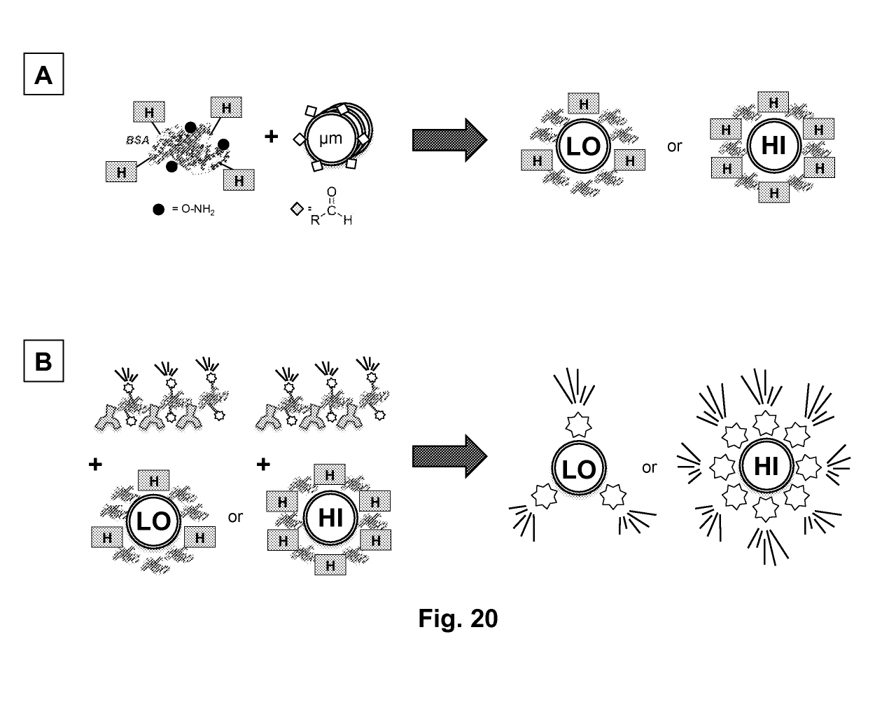

Preparation of haptenylated calibrant microspheres: Hapten-modified microspheres with controlled amounts of hapten were prepared from commercially available carboxy-modified microspheres internally labeled with a blue fluorescent tracking dye. The overall preparation procedure, which is illustrated schematically in FIG. 20A, includes the following steps: (1) conversion of the carboxyl groups to amines, (2) conversion of amino groups to 4FB (4-formylbenzamide) groups, (3) preparation of hapten-modified BSA, (4) immobilization of a controlled amount of hapten on the microsphere by modification of BSA with a hapten and immobilization of ratios of BSA-AOA/hapten-BSA-AOA mixtures on 4FB microspheres using aniline catalysis.

These steps may be carried out, for example, as follows:

Step 1: Conversion of carboxy-microspheres to amino-microspheres. Glacial blue COMPEL microspheres (700 μL of a 10 mg/mL solution; Bangs Labs, Fishers, Ind.) were washed twice with 100 mM MES, 0.9% NaCl, pH 5.8. The microspheres were re-suspended into MES buffer at a microsphere concentration of 50 mg/mL and EDC (23 μL of a 130 mg/mL solution in water) and sulfo-NHS (100 μL of a 30 mg/mL solution) were added. The tube was vortexed and placed on a rotator for 20 min. The microspheres were washed twice with MES buffer. To the microspheres suspended in MES buffer was added 0.5 M EDA (1.0 mL in 100 mM borate, pH 7.8-8.0) and the tube was placed on the rotator for 2 hours. The microspheres were washed with DI water/0.05% Tween three times then three times with Modification Buffer and used directly in the next step.
Step 2: Conversion of amino-microspheres to 4FB microspheres. To the amino-microspheres (˜40 mg/mL) in Modification Buffer was added sulfo-NHS-4FB (3.8 mg) and the reaction mixture was placed on the rotator for 2 hours. The microspheres were subsequently washed three times with DI water/0.05% Tween then four times with Conjugation Buffer. Conjugation Buffer was added to prepare a 25 mg/mL microsphere mixture.
Step 3: Hapten modification of BSA. To a solution of BSA in Modification Buffer (50 μL of a 8.0 mg/mL solution; 0.6 nmol) was added NHS-Peg4-DNP (4.48 μL of a 5.0 mg/mL solution in DMSO; 7 mol equiv) (Quantabiodesign, Columbus, Ohio). The reaction mixture was incubated at room temperature for 2 hours and desalted into Modification Buffer using a 0.5 mL Zeba column pre-equilibrated with Modification Buffer. The final protein concentration and number of DNP groups/BSA was determined by measuring the A₂₈₀ and A₃₅₀ (nm) absorptions. The measurements indicated quantitative recovery of BSA and 4.3 DNP/BSA. BSA has been similarly modified with digoxigen (ChromaLink Digoxigenin, Solulink, San Diego, Calif.) and biotin (ChromaLink Biotin, Solulink).
Step 4A: AOA modification of hapten-BSA/BSA mixtures. Three DNP-BSA/BSA ratios (6/4, 1/9 and 0/10 (control)) were prepared at 7.0 mg/mL final BSA concentration. To each ratio (0.1 mg; 14.3 μL in Modification buffer was added AOA-NHS conjugating reagent (1.93 μL of a 5.0 mg/mL solution in DMSO; 15 mol equiv). The reaction mixtures were incubated at room temperature for 2 h and desalted into Conjugation Buffer using a 75 μL Zeba column pre-equilibrated with Conjugation Buffer. The AOA-modified BSA mixtures were used directly in the next step.
Step 4B: Immobilization of hapten-BSA/BSA-AOA on 4FB-microspheres: To three tubes containing a mixture of 4FB-microspheres (0.875 mg; 35 μL of a 25 mg/mL solution in Modification Buffer) was added BSA mixtures (50 μg each) and aniline buffer (3.5 μL) and the tubes were placed on the rotator overnight at room temperature. Following incubation the microspheres were washed three times with PBS and diluted to 5 mg/mL.

Labeling of haptenylated-microspheres with an [(anti-hapten-BSA)-fluorophore] immunopolymer, produced according to the methods disclosed herein, was added to hapten-surfaced microspheres at low or high surface densities.

Due to the highly efficient reaction of haptens with anti-hapten polymers, this labeling results in surface signaling of the microspheres at either low or high intensity. The process is illustrated graphically in FIG. 20B.

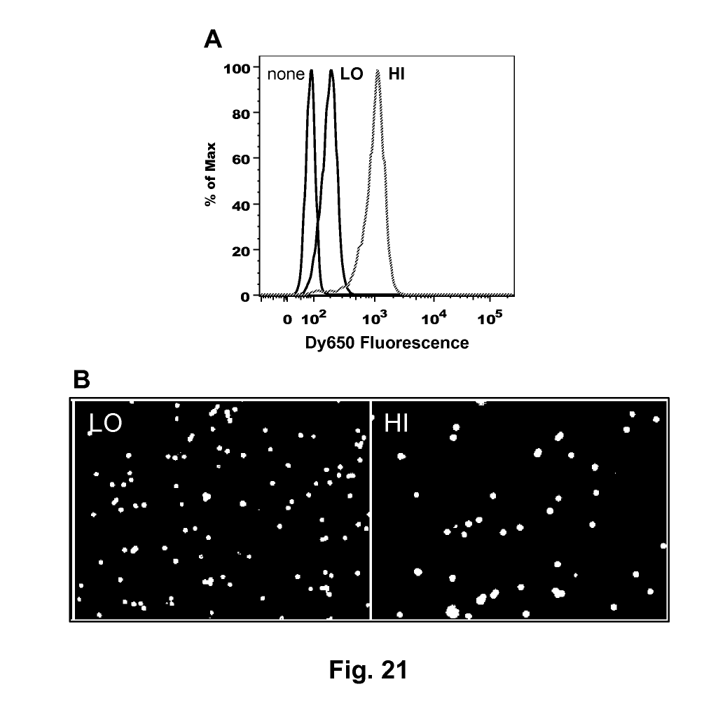

FIG. 21 shows laboratory evaluation of microspheres prepared as described above for use in flow cytometry and microscopic analyses. Here, DNP was used as the hapten, and an [(anti-DNP-BSA)-Dy650] immunopolymer was used as the fluorescent anti-hapten reagent.

FIG. 21A illustrates a flow cytometric evaluation of low-intensity (LO) and high-intensity (HI) microspheres using a standard laboratory cytometer (BD Fortessa), along with non-haptenylated microspheres (i.e., BSA-only surface, labeled ‘none’ in figure) to serve as a reference point. Results of the evaluation show an almost 10-fold difference in median signal intensity for LO vs HI microspheres when evaluated cytometrically.

FIG. 21B provides a microscopic evaluation of the haptenylated microspheres by viewing low-intensity (LO) and high-intensity (HI) microspheres through a cover glass-based microplate on a standard fluorescent microscope (Zeiss AxioVert 40CFL) at 20× magnification. Analysis settings (exposure, gain, etc.) were set using the signal of the high-intensity microspheres and were not changed during the viewing. The microscopic evaluation confirmed the cytometric results, with low-intensity microspheres appearing visibly dimmer than the high-intensity microspheres. This result was confirmed using quantitative imaging software, discussed in detail below. A sample containing blank microspheres emitted no visible signal (not shown).

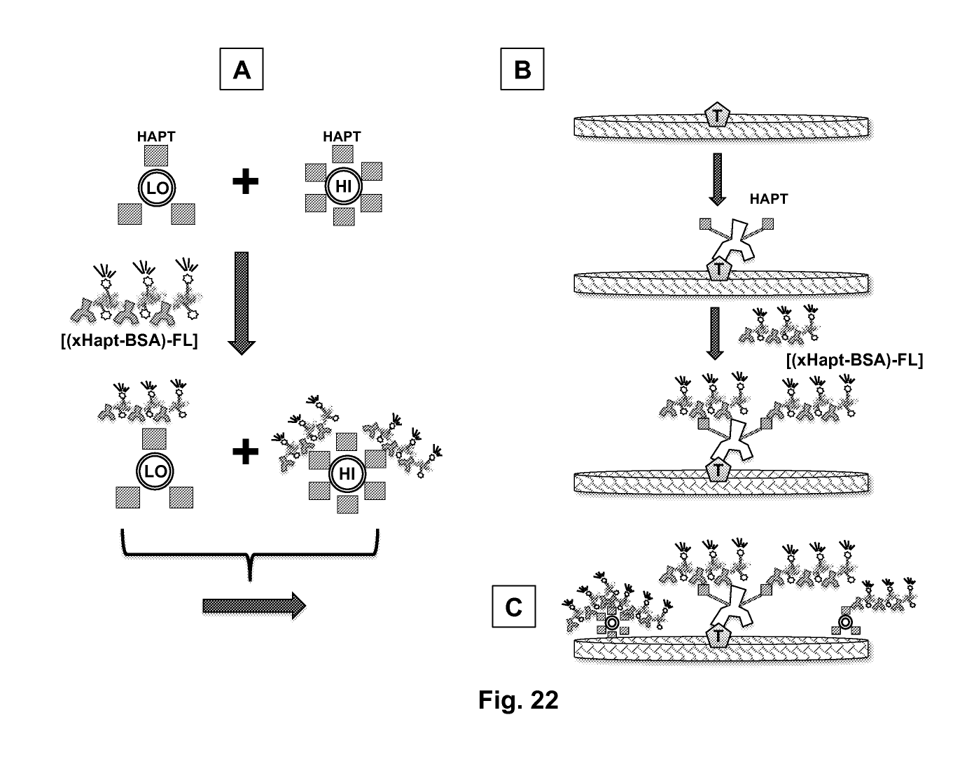

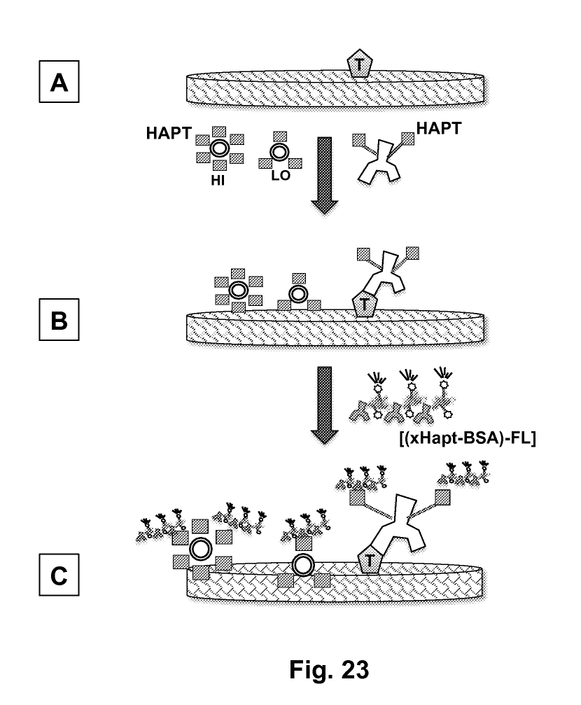

Use of the Calibrant Microspheres for Scoring of Tissue Samples: The haptenylated microspheres with different surface densities have been used as calibrants to enable automated scoring of labeled tissue samples. FIGS. 22 and 23 illustrate two exemplary workflows for utilization of the microspheres in conjunction with a tissue sample. As shown in FIG. 22, haptenylated microspheres with different surface densities of haptenylation may be pre-labeled with a fluorescent anti-hapten immunopolymer and then applied to tissue having been labeled using the same immunopolymer [(anti-hapten-BSA)-FL]. Alternatively, as shown in FIG. 23, haptenylated microspheres may be applied to a tissue slide without pre-labeling by the fluorescent anti-hapten immunopolymer. The tissue sample, which bears an antigen of interest, is first probed with a primary antibody bearing the same hapten as the microspheres. In FIG. 23, the antigen of interest is illustrated graphically as a pentagon, whereas the hapten is illustrated as a square. The fluorescent anti-hapten immunopolymer is then applied, thus labeling both the microspheres and the antigen of interest in the tissue. With either strategy, multiple antigens and multiple types of haptenylated microspheres may be analyzed in a single tissue sample, using multiple fluorescent channels.

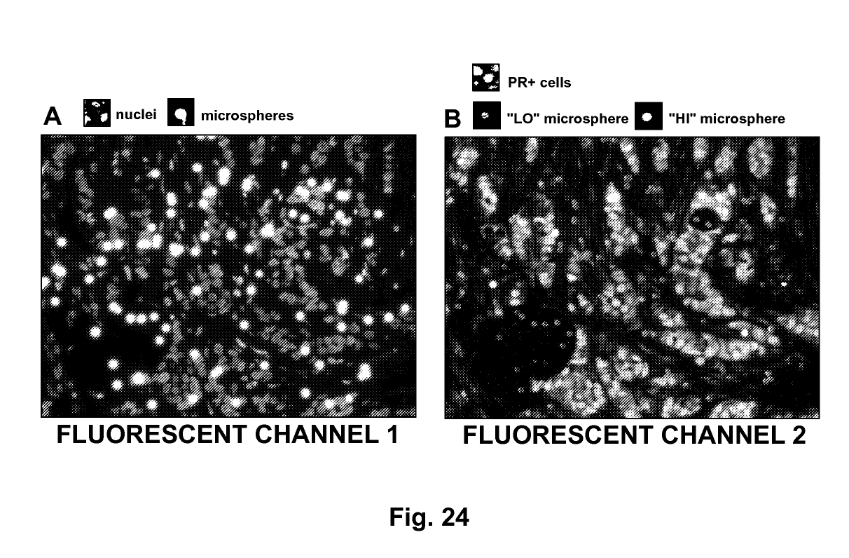

FIG. 24 shows the labeling method of FIG. 23 in use on a human FFPE breast biopsy tissue stained for a single antigen (progesterone receptor, PR). Specifically, haptenylated microspheres, pre-labeled with a fluorescent anti-hapten immunopolymer [(anti-DNP-BSA)-Dy650] were applied to a tissue already labeled with a haptenylated primary antibody anti-PR-DNP and the same fluorescent anti-hapten immunopolymer [(anti-DNP-BSA)-Dy650]. The stained samples were then coverslipped and imaged. (A) In fluorescent channel #1, the fluorescent tracking dye of the microspheres is visible, as well as nuclear counterstain DAPI. This information is used to enumerate all microspheres and all nuclei. (B) In fluorescent channel #2, ‘LO’ and ‘HI’ labeled calibrant microspheres are visible, as well as labeled cells of interest (here, PR+). This information is used to evaluate cellular signaling level, and enumerate cells of interest.

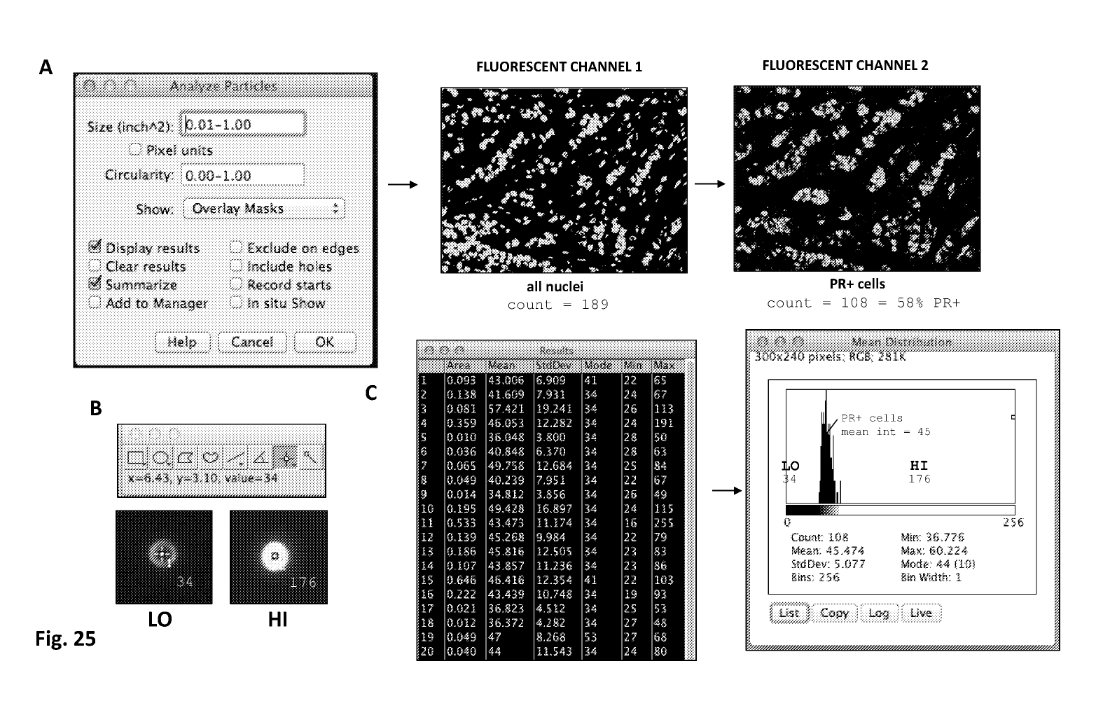

FIG. 25 illustrates an automated tissue analysis of the images of FIG. 24 using freely available imaging software (ImageJ, NIH). The auto-scoring setup proceeds as follows:

(A) Using the Particle Analysis function, the user sets up the software to identify and count nuclei and antigen-positive cells, allowing a percentage of antigen-positive cells to be calculated (here, PR+=58% total cells).
(B) By using the point-and-click tool on microspheres in the image, the emission intensity of LO and HI microspheres is scored by the software on a 256-point scale. Hereby, the user has calibrated the 256-point scale to identify low-signaling vs high-signaling antigen-positive cells.
(C) Single cell analysis is now run using the software. Antigen-positive status and signal intensity on the 256-point scale is recorded for each cell in the image field (left); these results can be displayed in a number of ways, but shown in the histogram (right) is the signal distribution of each PR+cell in the image field on the calibrated 256-point scale. Here, results indicate that the mean signal of the PR+ cells in this section of the tissue was very close to the low end of the scale.

Therefore, this tissue would be scored PR-LOW.

The analysis shown in FIG. 25 may be recorded as a “macro” (user-written program) and applied to hundreds of images uniformly and very quickly to score large areas of tissue.

The two parameters calculated as shown in FIG. 25 (percent antigen-positive and mean signal intensity) are used to calculate a well-known pathological diagnostic score, the Allred Score. Without stringent calibration of instrumentation and scoring as herein enabled by haptenylated microspheres, the Allred Score is subject to significant variation derived mostly from subjective scoring of staining intensity. The technology described herein enables the Allred Score, and similar accepted scoring methods, to be used with a minimum of user subjectivity, and a maximum of diagnostic efficiency and speed.

All patents, patent publications, and other published references mentioned herein are hereby incorporated by reference in their entireties as if each had been individually and specifically incorporated by reference herein.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined by reference to the appended claims, along with their full scope of equivalents.

1-75. (canceled)

76. A reagent mixture for calibration of an immunochemical assay, comprising:
a first population and a second population of particles comprising a detectable agent,
wherein the detectable agent is associated with the surface of the first population of particles at a first surface density and is associated with the surface of the second population of particles at a second surface density, and wherein the detectable agent is a cellular antigen or a hapten.

77. The reagent mixture of claim 76, wherein the particles are microspheres.

78. The reagent mixture of claim 76, further comprising a detectable immunoreagent specific for the detectable agent.

79. The reagent mixture of claim 78, wherein the detectable immunoreagent comprises a fluorescent label.

80. The reagent mixture of claim 78, wherein the detectable immunoreagent is a high-affinity immunopolymer comprising:
a plurality of antibodies;
a plurality of coupling proteins; and
a plurality of detectable labels;
wherein the plurality of antibodies and the plurality of coupling proteins are associated by a high-efficiency conjugation moiety.

81. The reagent mixture of claim 76, wherein the hapten is selected from the group consisting of: a nitrophenyl, a dinitrophenyl, a digoxygenin, a biotin, a Myc tag, a FLAG tag, an HA tag, an S tag, a Streptag, a His tag, a V5 tag, a ReAsh tag, a FlAsh tag, a biotinylation tag, Sfp tag, or a peptide tag.

82. A method for calibration of an immunochemical assay comprising:
treating a population of cells with a first population and a second population of particles comprising a detectable agent, wherein the detectable agent is associated with the surface of the first population of particles at a first surface density and is associated with the surface of the second population of particles at a second surface density, and wherein the detectable agent is a cellular antigen or a hapten.

83. The method of claim 82, wherein the particles are microspheres.

84. The method of claim 82, wherein the cells and the particles are also treated with a detectable immunoreagent specific for the detectable agent.

85. The method of claim 84, wherein the detectable immunoreagent comprises a fluorescent label.

86. The method of claim 84, wherein the detectable immunoreagent is a high-affinity immunopolymer comprising:
a plurality of antibodies;
a plurality of coupling proteins; and
a plurality of detectable labels;
wherein the plurality of antibodies and the plurality of coupling proteins are associated by a high-efficiency conjugation moiety.

87. The method of claim 84, wherein the cells and the particles are separately treated with the detectable immunoreagent.

88. The method of claim 84, wherein the cells and the particles are jointly treated with the detectable immunoreagent.

89-93. (canceled)

94. The reagent mixture of claim 76, wherein the reagent mixture comprises at least three populations of calibrant particles, each population with a different surface density of the detectable agent.

95. The method of claim 82, wherein the population of cells is treated with a third population of particles comprising the detectable agent associated with the surface of the third population of particles at a third surface density.

96. The method of claim 82, further including the step of detecting the amount of detectable agent in the cells and in the particles.

97. The method of claim 82, further including the step of scoring the level of detectable agent in the cells, based on the amount of detectable agent in the different populations of particles.

98. The method of claim 97, wherein the scoring step is performed automatically.
